SEQUENCE LISTING (18 sequences)

SEQ ID NO: 1 - protein, 40 aa, Homo sapiens
RIYKGVIQAIQKSDEGHPFRAYLESEVAISEELVQKYSNS

SEQ ID NO: 2 - protein, 25 aa, Homo sapiens
IQKSDEGHPFRAYLESEVAISEELV

SEQ ID NO: 3 - protein, 25 aa, Homo sapiens
RAYLESEVAISEELVQKYSNSALGH

SEQ ID NO: 4 - protein, 25 aa, Homo sapiens
SEELVQKYSNSALGHVNCTIKELRR

SEQ ID NO: 5 - protein, 25 aa, Homo sapiens
ALGHVNCTIKELRRLFLVDDLVDSL

SEQ ID NO: 6 - protein, 40 aa, Homo sapiens; identical to SEQ ID NO: 1

SEQ ID NO: 7 - protein, 66 aa, Homo sapiens
FRIYKGVIQAIQKSDEGHPFRAYLESEVAISEELVQKYSNSALGHVNCTIKELRRLFLVDDLVDSL

SEQ ID NO: 8 - protein, 473 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 9 - protein, 473 aa, Mus musculus
MKRASSGGSRLLAWVLWLQAWRVATPCPGACVCYNEPKVTTSCPQQGLQAVPTGIPASSQRIFLHGNRISHVPAASFQSCRNLTILWLHSNALARIDAAAFTGLTLLEQLDLSDNAQLHVVDPTTFHGLGHLHTLHLDRCGLRELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLMPLRSLQYLRLNDNPWVCDCRARPLWAWLQKFRGSSSEVPCNLPQRLADRDLKRLAASDLEGCAVASGPFRPIQTSQLTDEELLSLPKCCQPDAADKASVLEPGRPASAGNALKGRVPPGDTPPGNGSGPRHINDSPFGTLPSSAEPPLTALRPGGSEPPGLPTTGPRRRPGCSRKNRTRSHCRLGQAGSGASGTGDAEGSGALPALACSLAPLGLALVLWTVLGPC

SEQ ID NO: 10 - protein, 344 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 11 - protein, 310 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCA

SEQ ID NO: 12 - protein, 344 aa, Rattus norvegicus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 13 - protein, 310 aa, Rattus norvegicus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCA

SEQ ID NO: 14 - protein, 66 aa, Homo sapiens
RIYKGVIQAIQKSDEGHPFRAYLESEVAISEELVQKYSNSALGHVNCTIKELRRLFLVDDLVDSLK

SEQ ID NO: 15 - protein, 40 aa, Homo sapiens; identical to SEQ ID NO: 1

SEQ ID NO: 16 - protein, 37 aa, Homo sapiens
RIYKGVIQKSDEGHPFRAYLESEVAISEELVQKYSNS

SEQ ID NO: 17 - DNA, 33 bp, Artificial Sequence
  Description of Artificial Sequence Primer
tatggctagctatgcacatactttcacagaatt

SEQ ID NO: 18 - DNA, 35 bp, Artificial Sequence
  Description of Artificial Sequence Primer
ctatttaaatatcattgctgtaatcataatttgtc

(19) United States
(12) Patent Application Publication
(10) Pub. No.: US 2008/0274077 A1
(43) Pub. Date: Nov. 6, 2008

(54) Method for Treating Neurological Disorders

(75) Inventors: Larry I. Benowitz, Newton, MA (US); Dietmar Fischer, Dornstadt-Tomerdingen (DE)

Correspondence Address: David S Resnick;Nixon Peabody, 100 Summer Street, Boston, MA, 02110-2131, US

(73) Assignee: Children's Medical Center Corporation, Boston (US)

(21) Appl. No.: 10/580,364

(22) PCT Filed: Dec. 16, 2004
(86) PCT No.: PCT/US04/42255

§ 371 Date: Dec. 14, 2006

Related U.S. Application Data
(60) Provisional application No. 60/529,833, filed on Dec. 16, 2003.

(51) Int. Cl. A61K 38/00; A61K 38/18; A61K 38/20; A61K 31/11; A61K 31/351; A61K 31/665; A61K 38/46; A61K 38/45; A61K 39/395; A61K 38/16; A61K 31/70; A61P 25/00; A61K 31/708

(52) U.S. Cl. 424/85.2; 514/12; 514/693; 514/460; 514/99; 424/94.6; 424/94.5; 424/130.1; 514/44; 514/45

(57) ABSTRACT

The present invention is based on the discovery that suppressing the activity of the Nogo receptor (NgR) alone does not result in extensive axon regeneration unless the intrinsic growth program of neurons is also activated Accordingly, the present invention is directed to methods of stimulating axon regeneration using a combination therapy wherein agents that inhibit NgR activity or downstream pathways activated by inhibitory signals are combined with agents that activate the growth pathway of neurons (e.g. polypeptide growth factors, activators of macrophages, purine nucleosides, or hexoses).

### CROSS REFERENCE

This Application claims the benefit under 35 U.S.C § 119(e) of U.S. Provisional Application No. 60/529,833 filed Dec. 16, 2003.

### GOVERNMENT SUPPORT

The work described herein was supported, in part, by National Institute of Health grant No. EY05690. The U.S. Government has certain rights to the invention.

### BACKGROUND OF THE INVENTION

The inability of CNS neurons to regenerate their axons after injury places severe limitations on the functional recovery that can occur after traumatic injury, stroke, or certain neurodegenerative diseases. Regenerative failure has been attributed in part to proteins associated with CNS myelin and with glial scar that forms at an injury site. Several myelin inhibitors of axon growth, including the C-terminal of NogoA (Chen et al., 2000; GrandPre et al., 2000), myelin-associated glycoprotein, (McKerracher et al., 1994; Mukhopadhyay et al., 1994), and OMgp (Wang et al., 2002b), exert their effects via the Nogo receptor (NgR) and p75^{NTR} or another co-receptor (Fournier et al., 2001; Domeniconi et al., 2002; Liu et al., 2002; Wang et al., 2002a,b). In culture, expression of NgR causes growth cones of embryonic chick retinal ganglion cells (RGCs) to collapse upon contact with the C-terminal region of Nogo (Nogo66) (Fournier et al., 2001) and inhibits neurite outgrowth from cerebellar granule cells on MAG, OMgp, or myelin (Wang et al., 2002a,b). Conversely, transfection with dominant-negative form of NgR (NgR^{DN}) enables cerebellar granule cells in culture to overcome the inhibitory effects of myelin, Nogo66, OMgp, and MAG (Domeniconi et al., 2002; Wang et al., 2002a,b). However, the effects of overexpressing either NgR or NgR^{DN} have not been investigated in vivo, nor have the effects of deleting the gene.

Antibodies to NogoA, or a small peptide inhibitor of NgR, increase corticospinal tract (CST) regeneration only to some extent in rats (Schnell et al., 1994; Bregman et al., 1995; GrandPre et al., 2002; Sicotte et al. 2003), whereas genetic deletion of the NogoA gene in mice results either in a modest CST regeneration (Kim et al., 2003b; Simonen et al., 2003) or in none (Zheng et al., 2003). Thus, overcoming specific myelin inhibitors, or suppression of signaling through NgR, is not sufficient to promote the substantive CNS regeneration in vivo that would be required for the treatment of neurological disorders (Steward et al., 2003; Woolf, 2003; Zheng et al., 2003).

There is a need in the art for methods and compositions that can improve the ability of a neuron, or portion of the nervous system, to regenerate, and to maintain desirable function, which can be used for treatment of neurological disorders.

### SUMMARY OF THE INVENTION

The present invention is based on the discovery that suppressing the activity of the Nogo receptor (NgR) alone does not result in extensive axon regeneration unless the innate growth pathway of neurons is also activated. Accordingly, the present invention is directed to methods of stimulating axon regeneration using a combination therapy wherein agents that inhibit NgR activity are combined with agents that activate the growth pathway of neurons (e.g. polypeptide growth factors, e.g., BDNF, CNTF, NGF, IL-6, GDNF; activators of macrophages, such as GM-CSF, TGF-β; growth factors produced by macrophages, e.g., oncomodulin or MIF; purine nucleosides, such as inosine; or hexoses, such as mannose).

In one embodiment, a method for stimulating the axonal growth of central nervous system (CNS) neurons is provided comprising the steps of i) contacting CNS neurons with an effective amount of an NgR antagonist; and ii) contacting CNS neurons with an effective amount of an agent that activates the growth pathway of CNS neurons.

Neurons can be contacted with each agent either separately or simultaneously. In one preferred embodiment, neurons are contacted with an agent that activates the growth pathway of CNS neurons prior to contacting with an NgR antagonist.

Examples of suitable agents that can be used for activation of the growth pathway of CNS neurons in the present invention include, but are not limited to, inosine, oncomodulin, known polypeptide growth factors such as NGF, NT-3, NGF, CNTF, IL-6, GDNF, TGF-β and hexose molecules, such as D-mannose, gulose and glucose-6-phosphate.

In one aspect, the method for stimulating the axonal growth of central nervous system (CNS) neurons, as described herein, further comprises contacting CNS neurons with a cAMP modulator that increases the concentration of intracellular cAMP. Suitable cAMP modulators for use in the present invention include, but are not limited to cAMP analogues, activators of G protein coupled receptors that activate cAMP, adenylate cyclase activators, calcium ionophores, and phosphodiesterase inhibitors.

Suitable NgR antagonist for use in the present invention include any agent able to suppress the activity of the Nogo receptor. For example, the NgR antagonist can be an agent that binds to the Nogo receptor thereby inhibiting signaling mediated by NgR, an agent that binds to a ligand of NgR (e.g. OMgp, MAG, or NOGO) thereby inhibiting binding of the ligand to NgR, an agent that inhibits the expression of NgR, or an agent that inhibits the activity of a downstream signaling molecule that is activated by NgR, such as RhoA or Rho kinase (ROCK). NgR antagonists can be antibodies, peptides, a small molecules, RNAs (e.g. siRNA or antisense-RNA), or DNAs.

In the methods described herein, any combination of an NgR antagonist and an agent that activates the growth pathway of CNS neurons can be used.

In one embodiment, the NgR antagonist is a peptide that binds to NgR, said peptide being selected from the group consisting of SEQ ID NO:1, SEQ ID NO: 2, SEQ ID NO: 3, SEQ ID NO: 4. SEQ ID NO: 5, SEQ ID NO: 6 and SEQ ID NO: 7.

In one embodiment, the NgR antagonist is a peptide that comprises the amino acid residues of human NogoA set forth in SEQ ID NO: 14.

In one embodiment, the NgR antagonist is a peptide that comprises the amino acid residues of human NogoA forth in SEQ ID NO:15.

In one embodiment, the NgR antagonist is a peptide that comprises the amino acid sequence of Nogo-66 set forth in SEQ ID NO:16.

In another embodiment, the NgR antagonist is a soluble NgR protein.

In one embodiment, the soluble NgR protein comprises the amino acid sequence set forth in SEQ ID NO: 8 or in SEQ ID NO: 9.

In one embodiment, the soluble NgR protein is a soluble Nogo Receptor-1 polypeptide sequence selected from the group consisting of amino acid residues 26-344 of SEQ ID NO: 10; amino acid residues 26-310 of SEQ ID NO: 11; amino acid residues 26-344 of SEQ ID NO: 12; amino acid residues 27-344 of SEQ ID NO: 12; and amino acid residues 27-310 of SEQ ID NO: 13.

In another embodiment, the NgR antagonist is a nucleic acid aptamer that binds to NgR.

In one embodiment, the NgR antagonist is a DNA that encodes a dominant negative form of NgR. The DNA can be contained in a viral vector (e.g. AAV) whereby administration of said vector is a means for contacting CNS neurons with an effective amount of NgR antagonist. Any viral vector can be used in the methods of the present invention.

In one embodiment, the NgR antagonist is an agent that inhibits the activity of a downstream signaling molecule that is activated by NgR, such as clostridium botulinum C3 ADP-ribosyltransferase that inhibits the downstream signaling molecule RhoA.

In another embodiment, a method for treating a neurological disorder in a patient is provided that comprises the steps of i) administering an effective amount of an NgR antagonist to a patient; and ii) administering to said patient an effective amount of an agent that activates the growth pathway of CNS neurons.

Any neurological disorder that would benefit from new axonal growth can be treated by the methods of the present invention.

In one embodiment, the neurological disorder to be treated is selected from the following: traumatic brain injury, stroke, cerebral aneurism, spinal cord injury, Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, opthalmoplegia, and retina or optic nerve damage.

Pharmaceutical compositions comprising a NgR antagonist and an agent that activates the growth pathway of CNS neurons is also provided. The composition is formulated for administration, including, for example topical, pulmonary, internal topical, interdermal, parenteral, subcutaneous, intranasal, epidermal, ophthalmic, oral, intraventricular, and intrathecal administration.

In one embodiment, the invention includes a kit having a container of an NgR antagonist and a container of an agent that activates the growth pathway of CNS neurons.

### BRIEF DESCRIPTION OF FIGURES

FIG. 1 shows quantization of axon regeneration and RGC survival. A: Quantization of axon growth at 0.5 mm (light bars) and 1 mm (dark bars) distal to the injury site. B: Cell survival (βIII tubulin-positive RGCs per section). ††decrease relative to GFP-transfected controls significant at p<0.01; **increase relative to GFP-transfected controls significant at p<0.01.

FIG. 2 shows axon regeneration on permissive and non-permissive substrates. A-B: Retinal explants grown on a permissive laminin/poly-L-lysine substrate. A: Quantization of axon growth. Control retina not exposed to macrophage-derived factors in vivo (i.e., no lens injury) and in retinas transfected with AAV-NgR^{WT}-IGFP and exposed to macrophage-derived factors in vivo or axons arising from growth-activated retina transfected with AAV-NgR^{DN}-IGFP B: Growth of transfected retinal explants (exposed to macrophage-derived factors in vivo) on myelin (percentage of axons arising from explants that extend >500 μm). †††decrease relative to controls significant at p<0.001; **increase relative to controls significant at p<0.000. Scale bar: 100 μm.

FIG. 3 shows that activation of the growth pathway of RGCs and inactivation of RhoA have synergistic effects in vivo. GAP-43-positive axons visualized in longitudinal sections through the adult rat optic nerve 2 weeks after axotomy with or without lens injury. RGCs were transfected with AAV expressing GFP alone or C3 plus GFP .a, Absence of regeneration after axotomy alone. FIG. 3A, Quantitation of outgrowth (number of axons growing ≧500 μm beyond the injury site per optic nerve). FIG. 3B, RGC survival (TUJ1⁺ RGCs per retinal cross section). Axot, Axotomy; LI, lens injury. ***Effect of C3 expression significant at p<0.001. †††, effect of intravitreal macrophage activation significant at p<0.001. Scale bar, 200 μm.

FIG. 4 shows the effect RhoA inactivation on axon regeneration depends on growth state and substrate: in vitro studies. Retinal explants were grown on poly-L-lysine-laminin substrate without or with myelin proteins 2 weeks after transfecting RGCs in vivo with genes expressing GFP alone or C3 expression has a small stimulatory effect under these conditions. Optic nerve injury 4 d before explanting increases outgrowth slightly relative to controls and C3 expression enhances this growth considerably. Exposure of axotomized RGCs to the effects of lens injury increases outgrowth greatly, but C3 expression has no additional effect. Myelin proteins diminish outgrowth from growth-activated RCGs, and C3 expression partially reverses this inhibition. The graph shows the quantitation of results. Significance of C3 expression: **p<0.02; ***p<0.001; †††differences between experimental treatments significant at p<0.001. Scale bar, 250 μm.

FIG. 5 shows SEQ ID NO: 1, SEQ ID NO: 2, SEQ ID NO: 3, SEQ ID NO: 4, SEQ ID NO: 5, SEQ ID NO: 6, and SEQ ID NO: 7.

FIG. 6 shows SEQ ID NO: 8.

FIG. 7 shows SEQ ID NO: 9.

FIG. 8 shows SEQ ID NO: 10, SEQ ID NO: 11, SEQ ID NO: 12, and SEQ ID NO: 13.

FIG. 9 shows SEQ ID NO: 14, SEQ ID NO: 15, and SEQ ID NO: 16.

### DETAILED DESCRIPTION OF THE INVENTION

The present invention provides methods of stimulating axonal growth of central nervous system (CNS) neurons that can be used for treating neurological disorders. The methods presented herein use a combination therapy that involves stimulation of axonal growth by both i) activating the growth pathway of CNS neurons and, ii) inhibiting the activity of NgR using an antagonist of NgR. Pharmaceutical compositions comprising these agents are also included. Preferred compositions are formulated for intravenous or intrathecal administration.

### DEFINITIONS

The following definitions are provided for specific terms which are used in the following written description.

As used herein, the term “NgR antagonist” includes any agent that decreases, inhibits, blocks or interferes with NgR activity. The antagonist can be an agent that binds to NgR thereby inhibiting signal mediated by the receptor. Alternatively, the antagonist can be an agent that inhibits the expression of NgR, such as anti-sense RNA, or RNAi. The term antagonist, as used herein, also encompasses agents that inhibit the activity of a downstream signaling molecules that are activated by NgR, or the antagonist can be a dominant-negative form of NgR. Antagonists include, for example, antibodies, as defined herein, and molecules having antibody-like function such as synthetic analogues of antibodies, e.g., single-chain antigen binding molecules, small binding peptides, or mixtures thereof. Agents having antagonist activity can also include small organic molecules, natural products, peptides, aptamers, peptidomimetics, DNA and RNA.

Suitable NgR antagonists for use in methods of the invention include, but are not limited to, NEP1-40, a peptide antagonist which prevents NgR ligands from binding but which does not activate downstream signaling (Nature. 2002 May 30; 417(6888):547-51; J. Neurosci. 2003 May 15; 23(10):4219-27); monoclonal antibodies to the receptor (J Biol. Chem. 2004 Oct. 15; 279(42):43780-8) and those disclosed in WO 2004/014311, such as mAb's 7E11, 5B10, 1H2, 3G5, 2F7, ID9.3, 2G7.1, 1E4.1, 1G4.1, 2C4.1, 2F11.1, 1H4.1, 2E8.1, 2G11.2, and 1B5.1; soluble fusion proteins, consisting of the ligand-binding domain of the NgR receptor linked to part of an immunoglobulin (NgR(310)ecto-Fc), that binds to NgR ligands and prevent them from interacting with the receptor on axons (J Neurosci. 2004 Jul. 7; 24(27):6209-17; J Neurosci. 2004 November 17; 24(46):10511-20) and those disclosed in WO 2004/014311, such as sNogoR310 and sNogoR310-Fc and sNgR disclosed in MacDermid et al., 2004 European Journal of Neuroscience 20(10):p2567; soluble NgR, such as sNgR^{c-term} and sNgR^{c-term} as disclosed in WO 2004/090103; a dominant-negative form of the Nogo Receptor (Neuron. 2002 Jul. 18; 35(2):283-90; and J Neurosci. 2004 Feb. 18; 24(7):1646-51); clostridium botulinum C3 ADP-ribosyltransferase that inactivates RhoA; Y-27632, a small molecule inhibitor of ROCK (Dergham et al., 2002 J. Neurosci. 22: 6570-6577 and Lehmann et al. 1999 J. Neurosci. 19: 7537-7547); Nogo antagonist Pep2-41 and synthetic peptide 140 (PCT WO 03/031462; US 2002/0077295) and NEP1-40, a NgR antagonist 40 residue peptide that is commercially available from Phenix Pharmaceuticals Inc. (GrandPre et al., Nature 2002 417: 547-541), other NgR antagonist peptides are described in Fouiner et al., 2001 Nature 409: 341-346, Huber et al., 2000 Biol. Chem 381: 407-419, Oertle, T et al., 2003 J. Neurosci. 23:5393-5406; and antibodies that block Nogo such as IN-1 antibody (Brosamle et al., J. Neurosci 2000 20: 8061-8068) and 7B12 (Wiessner et al., 2003 J. Cereb. Blood Flow Metab. 23: 154-165) as well as others, such as described in Schnell et al., Nature. 1990 Jan. 18; 343(6255):269-72; Kapfhammer et al., J Neurosci. 1992 June; 12(6):2112-9; Guest et al., J Neurosci Res. 1997 Dec. 1; 50(5):888-905; Z'Graggen et al., Neurosci. 1998 Jun. 15; 18(12):4744-57; Bareyre et al., J Neurosci. 2002 Aug. 15; 22(16):7097-110; and Fouad et al., Eur J Neurosci. 2004 November; 20(9):2479-82.

In one embodiment, the NgR antagonist comprises a peptide that binds to the NgR selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

In another embodiment, the NgR antagonist is a soluble NgR protein comprising the amino acid sequence of SEQ ID NO: 8 or SEQ ID NO: 9. In some embodiments, the soluble NgR is a fusion protein, e.g., an Fc-fusion protein. In some embodiments, the invention provides a soluble Nogo receptor-1 polypeptide consisting essentially of a N-terminal domain (NT), 8 leucine rich repeat domains (LRR) and a LRR C-terminal domain (LRRCT) of Nogo receptor 1. In some embodiments, said soluble Nogo receptor-1 polypeptide is joined to a signal sequence. In some embodiments, the LRR comprises a heterlogous LRRR. In some embodiments, the invention provides a soluble Nogo receptor-1 polypeptide selected from the group consisting of: amino acid residues 26-344 of SEQ ID NO: 10; amino acid residues 26-310 of SEQ ID NO: 11; amino acid residues 26-344 of SEQ ID NO: 12; amino acid residues 27-344 of SEQ ID NO: 12; and amino acid residues 27-310 of SEQ ID NO: 13.

In one embodiment, the NgR antagonist peptide 140 (amino acid residues of 1055-1120 of human NogoA; see US 2002/0077295), which comprises SEQ ID NO: 14 that is acetylated at the C-terminus and amidated at the N-terminus.

In another embodiment, the NgR antagonist is Pep2-41 (amino acid residues 1055-1094 of human NogoA; see PCT Publication WO 03/031462), which comprises SEQ ID NO: 15 that is acetylated at the C-terminus and amidated at the N-terminus.

In another embodiment, the NgR antagonist is NEP1-40 (see GrandPre et al., Nature 2002 417: 547-541), which comprises SEQ ID NO: 16.

In some embodiments, the NgR antagonist is a nucleic acid aptamer that binds to a Nogo Receptor, or a portion thereof, and disrupts interaction of NOGO with the NOGO receptor. Preferred aptamers are disclosed in U.S. 2003/0203870.

As used herein, the term “antibody”, includes human and animal mAbs, and preparations of polyclonal antibodies, as well as antibody fragments (antigen binding fragments), synthetic antibodies, including recombinant antibodies (antisera), chimeric antibodies, including humanized antibodies, anti-idiotopic antibodies and derivatives thereof.

In some embodiments, the antibody or antigen-antibody fragment binds to the NgR and inhibits Nogo receptor binding to a ligand (anti-NgR antibody). In one embodiment, a monoclonal antibody to the receptor is selected from the group consisting of 7E11, 5B10, 1H2, 3G5, 2F7, ID9.3, 2G7.1, 1E4.1, 1G4.1, 2C4.1, 2F11.1, 1H4.1, 2E8.1, 2G11.2, and 1B5.1 (See WO 2004/014311).

In some embodiments, the antibody or antigen-antibody fragment binds to a NgR ligand, such as OMgp, Nogo or MAG. Preferred anti-OMgP antibody or antigen-antibody fragment binds are disclosed in U.S. 2003/0113325. Preferred antibodies that block Nogo include IN-1 antibody (Brosamle et al., J. Neurosci 2000 20: 8061-8068) and 7B12 (Wiessner et al., 2003 J. Cereb. Blood Flow Metab. 23: 154-165).

U.S. Application No. 2003/0113325 also discloses peptides that bind OMgp, which are useful NgR antagonists in methods of the invention.

As used herein, the term “hexose” includes any hexose, or derivative thereof, that is able to activate the growth pathway of CNS neurons. Preferred hexoses include D-mannose and gulose. The term “hexose derivative” refers to a hexose molecule that has one or more residues (e.g. esters, ethers, amino groups, amido groups, phosphate groups, sulphate groups, carboxyl groups, carboxy-alkyl groups, and combinations thereof) covalently or ionically attached to one or more of the molecules hydroxyl groups. A preferred derivative includes glucose-6-phosphate. The term hexose derivative includes D- and L-isomers of hexose or hexose derivatives able to activate the growth pathway of CNS neurons. Hexose derivatives are well known in the art and commercially available (See also, for example, WO 2004/028468).

As used herein, an agent that “activates the growth pathway of CNS neurons” refers to an agent that elicits a response or result favorable to the health or function of a CNS neuron. Examples of such effects include improvements in the ability of a neuron or portion of the nervous system to resist insult, to regenerate, to maintain desirable function, to grow or to survive.

As used herein, the term “cAMP modulator” includes any compound which has the ability to modulate the amount, production, concentration, activity or stability of cAMP in a cell, or to modulate the pharmacological activity of cellular cAMP. cAMP modulators may act at the level of adenylate cyclase, upstream of adenylate cyclase, or downstream of adenylate cyclase, such as at the level of cAMP itself, in the signaling pathway that leads to the production of cAMP. Cyclic AMP modulators may act inside the cell, for example at the level of a G-protein such as Gi, Go, Gq, Gs and Gt, or outside the cell, such as at the level of an extra-cellular receptor such as a G-protein coupled receptor. Cyclic AMP modulators include activators of adenylate cyclase such as forskolin; nonhydrolyzable analogues of cAMP including 8-bromo-cAMP, 8-chloro-cAMP, or dibutyryl cAMP (db-cAMP); isoprotenol; vasoactive intestinal peptide; calcium ionophores; membrane depolarization; macrophage-derived factors that stimulate cAMP; agents that stimulate macrophage activation such as zymosan or IFN-y; phosphodiesterase inhibitors such as pentoxifylline and theophylline; specific phosphodiesterase IV (PDE IV) inhibitors; and beta 2-adrenoreceptor agonists such as salbutamol. The term cAMP modulator also includes compounds which inhibit cAMP production, function, activity or stability, such as phosphodiesterases, such as cyclic nucleotide phosphodiesterase 3B. cAMP modulators which inhibit cAMP production, function, activity or stability are known in the art and are described in, for example, in Nano et al., Pflugers Arch 439 (5): 547-54, 2000, the contents of which are incorporated herein by reference.

Examples of phosphodiesterase IV inhibitors suitable for use in the present invention include, but are not limited to, 4-arylpyrrolidinones, such as rolipram (A. G. Scientific, Inc.), nitraquazone, denbufylline, tibenelast, CP-80633 and quinazolinediones such as CP-77059.

Examples of Beta-2 adrenoreceptor agonist suitable for use in the present invention include, but are not limited to, salmeterol, fenoterol and isoproterenol.

As used herein, the term “administering” to a patient includes dispensing, delivering or applying an active compound in a pharmaceutical formulation to a subject by any suitable route for delivery of the active compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route. The agents may, for example, be administered to a comatose, anesthetized or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

As used herein, the term “contacting CNS neurons” refers to any mode of agent delivery or “administration” either to cells, or to whole organisms in which the agent is capable of exhibiting it's pharmacological effect in neurons. “contacting CNS neurons” is intended to include both in vivo and in vitro methods of bringing an agent of the invention into proximity with a neuron. Suitable modes of administration can be determined by those skilled in the art and such modes of administration may vary between agents. For example, when axonal growth of CNS neurons is stimulated ex vivo, agents can be administered, for example, by transfection, lipofection, electroporation, viral vector infection, or by addition to growth medium. An in vivo means of contacting neurons with an agent that activates the growth pathway of neurons includes, but is not limited to, for example lens injury. Lens injury leads to macrophage activation and factors secreted from macrophages stimulate RGCs to regenerate their axons (Yin et al, 2003).

As used herein, “effective amount” of an agent is an amount sufficient to achieve a desired therapeutic or pharmacological effect, such as an amount sufficient to inhibit the activity of NgR, or an amount that is capable of activating the growth pathway of CNS neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

A therapeutically effective amount or dosage of an agent may range from about 0.001 to 30 mg/kg body weight, with other ranges of the invention including about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, and 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an active compound can include a single treatment or a series of treatments. In one example, a subject is treated with an agent in the range of between about 0.1 to 20 mg/kg body weight, one time per week for between about 1 to 10 weeks, alternatively between 2 to 8 weeks, between about 3 to 7 weeks, or for about 4, 5, or 6 weeks. It will also be appreciated that the effective dosage of an agent used for treatment may increase or decrease over the course of a particular treatment. The agents of the present invention can be administered simultaneously or separately.

As used herein, the term “patient” or “subject” or “animal” or “host” refers to any mammal. The patient is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the term “Neurological disorder” is intended to include a disease, disorder, or condition which directly or indirectly affects the normal functioning or anatomy of a subject's nervous system.

As used herein, the term axonal “growth” or “outgrowth” includes the process by which axons or dendrites extend from a neuron. The outgrowth can result in a new neuritic projection or in the extension of a previously existing cellular process. Axonal outgrowth may include linear extension of an axonal process by 5 cell diameters or more. Neuronal growth processes, including neuritogenesis, can be evidenced by GAP-43 expression detected by methods such as immunostaining. “Stimulating axonal growth” means promoting axonal outgrowth.

As used herein, the term “CNS neurons” is intended to include the neurons of the brain, the cranial nerves and the spinal cord.

As used herein, “NgR” refers to a receptor that binds to Nogo, or to isoforms of Nogo. For example, Nogo-66 (Fournier et al., 2001, Nature, 409(6818):341-346). Non-limiting examples of Nogo receptors are found in Genebank at accession numbers NM_{—}181377.2, AY311478.1, NM_{—}181380.2, AF462390.1, NM_{—}178570.1, NM_{—}178568.1, AF283463.1, and AF532858. Several Nogo Receptor homologues are also described in U.S. patent applications 20030124704, and 0020077295, which are herein incorporated by reference in their entirety. The term “NgR” is also intended to encompass homologues and allelic variants thereof.

Various aspects of the invention are described in further detail in the following subsections:

### NgR Antagonists

The combination therapy described herein comprises contacting CNS neurons with a NgR antagonist. The NgR antagonist can be administered before, concurrently with, or after administration of the agent that activates the growth pathway of CNS neurons. When the antagonist of NgR and additional therapeutic agent are administered at different times, they are preferably administered within a suitable time period to provide substantial overlap of the pharmacological activity of the agents. The skilled artisan will be able to determine the appropriate timing for co-administration of an antagonist and the additional agent depending on the particular agents selected and other factors.

The NgR antagonist can be DNA, RNA, a small organic molecule, a natural product, protein (e.g., antibody), peptide or peptidomimetic. Antagonists can be identified, for example, by screening libraries or collections of molecules, such as, the Chemical Repository of the National Cancer Institute, as described herein or using other suitable methods. Suitable screening methods that can be used to identify NgR antagonists for use in the present invention, as well as known NgR antagonists are described in U.S. Patent Application Nos. 20030203870, 20030186267, 20030113891, 20030113326, 20030113325, 20030060611, 20020077295, 20020012965, 2003/0113325, and PCT publication WO 2004/014311, which are herein incorporated by reference in their entirety. In particular, U.S. Application Nos. 20030186267, 20030113891, and 20030060611 describe ribozymes that cleave NgR mRNA and anti-sense molecules.

Another source of antagonists is combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

The term “peptide”, as used herein, refers to a compound consisting of from about two to about ninety amino acid residues wherein the amino group of one amino acid is linked to the carboxyl group of another amino acid by a peptide bond.

A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitrulline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can antagonize NgR function. Such peptide antagonists can then be isolated by suitable means.

The term “peptidomimetic”, as used herein, refers to molecules which are not polypeptides, but which mimic aspects of their structures. For example, polysaccharides can be prepared that have the same functional groups as peptides which can antagonize NgR. Peptidomimetics can be designed, for example, by establishing the three dimensional structure of a peptide agent in the environment in which it is bound or will bind to NgR. The peptidomimetic comprises at least two components, the binding moiety or moieties and the backbone or supporting structure.

The binding moieties are the chemical atoms or groups which will react or form a complex (e.g., through hydrophobic or ionic interactions) with NgR, for example, with the amino acid (s) at or near the ligand binding site. For example, the binding moieties in a peptidomimetic can be the same as those in a peptide antagonist of NgR. The binding moieties can be an atom or chemical group which reacts with the receptor in the same or similar manner as the binding moiety in a peptide antagonist of NgR. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more-CONH-groups for a-NHCO-group). In yet another embodiment, the peptide backbone can be substituted with a polysilane backbone.

These compounds can be manufactured by known methods. For example, a polyester peptidomimetic can be prepared by substituting a hydroxyl group for the corresponding a-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Peptidomimetics can be synthesized and assembled into libraries comprising a few to many discrete molecular species. Such libraries can be prepared using well known methods of combinatorial chemistry, and can be screened as described herein to determine if the library comprises one or more peptidomimetics which antagonize NgR function. Such peptidomimetic antagonists can then be isolated by suitable methods.

As used herein, an “antibody that inhibits NgR activity” or “anti-NgR antibody” includes an antibody or antigen-binding fragment. The term “antibody” as used herein encompasses polyclonal or monoclonal antibodies as well as functional fragments of antibodies, including fragments of chimeric, human, humanized, primatized, veneered or single-chain antibodies. Functional fragments include antigen-binding fragments which bind to NgR. For example, antibody fragments capable of binding to NgR or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Single-chain antibodies, and chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19 (9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for mammalian (e.g., human) NgR can be raised against an appropriate immunogen, such as isolated and/or recombinant human NgR or portions thereof (including synthetic molecules, such as synthetic peptides).

Preparation of immunizing antigen, and polyclonal and monoclonal antibody production can be performed using any suitable technique. For example, monoclonal antibodies directed against binding cell surface epitopes can be readily produced by one skilled in the art. The general methodology for making monoclonal antibodies by hybridomas is well known. Other suitable methods of producing or isolating antibodies of the requisite specificity can be used, including, for example, methods which select recombinant antibody from a library (e.g., a phage display library). Transgenic animals capable of producing a repertoire of human antibodies (e.g., XenoMouse™ (Abgenix, Fremont, Calif.)) can be produced using suitable methods (see, e.g., WO 98/24893 (Abgenix), published Jun. 11, 1998; Kucherlapati, R. and Jakobovits, A., U.S. Pat. No. 5,939,598; Jakobovits et al., Proc. Natl. Acad. Sci. USA, 90: 2551-2555 (1993); Jakobovits et al., Nature, 362: 255-258 (1993)). Additional methods for production of transgenic animals capable of producing a repertoire of human antibodies have been described (e.g., Lonberg et al., U.S. Pat. No. 5,545,806; Surani et al., U.S. Pat. No. 5,545,807; Lonberg et al., WO97/13852).

The NgR antagonist of the invention can also be an RNA interfering agent, such as siRNA The use of siRNAs and siRNA-based technologies (for example, shRNA-expression vectors) has proven to be a powerful tool for the silencing of gene expression in a sequence-specific manner and has been found to be amenable to a wide variety of mammalian cell types and tissues. Not only have siRNAs proven to be effective for the dissection of gene function, their application as a therapeutic modality is being aggressively investigated.

### Delivery of RNA Interfering Agents

In one embodiment, the RNA interfering agents used in the methods of the invention, e.g., the siRNAs, are taken up actively by cells in vivo following intravenous injection, e.g., hydrodynamic injection, without the use of a vector.

Other strategies for delivery of the RNA interfering agents, e.g., the siRNAs or shRNAs used in the methods of the invention, may also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

In one embodiment, the dsRNA, such as siRNA or shRNA, is delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the RNA interfering agents, e.g., the siRNAs used in the methods of the invention, can be introduced into cells, e.g., cultured cells, which are subsequently transplanted into the subject by, e.g., transplanting or grafting, or alternatively, can be obtained from a donor (i.e., a source other than the ultimate recipient), and applied to a recipient by, e.g., transplanting or grafting, subsequent to administration of the RNA interfering agents, e.g., the siRNAs of the invention, to the cells. Alternatively, the RNA interfering agents, e.g., the siRNAs of the invention, can be introduced directly into the subject in such a manner that they are directed to and taken up by the target cells and regulate or promote RNA interference of NgR expression. The RNA interfering agents, e.g., the siRNAs of the invention, may be delivered singly, or in combination with other RNA interfering agents.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi).

Preferably, the RNA interfering agent in the methods of the present invention is siRNA.

The NgR targeting siRNAs are designed so as to maximize the uptake of the antisense (guide) strand of the siRNA into RNA-induced silencing complex (RISC) and thereby maximize the ability of RISC to target NGR mRNA for degradation. This can be accomplished by looking for sequences that has the lowest free energy of binding at the 5′-terminus of the antisense strand. The lower free energy would lead to an enhancement of the unwinding of the 5′-end of the antisense strand of the siRNA duplex, thereby ensuring that the antisense strand will be taken up by RISC and direct the sequence-specific cleavage of NgR mRNA.

### RNA Interfering Agents

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent may be a cellular gene or genomic sequence. An siRNA may be substantially homologous to the target gene or genomic sequence, or a fragment thereof. As used herein, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target allele so as to prevent its interaction with the normal allele.

The siRNA preferably targets only one sequence. Each of the RNA interfering agents, such as siRNAs, can be screened for potential off-target effects may be analyzed using, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology.

Agents that Activate the Growth Pathway of CNS Neurons

Agents that activate the growth pathway of CNS neurons are agents that are capable of producing a neurosalutary effect. As used herein, a “neurosalutary effect” means a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The phrase “producing a neurosalutary effect” includes producing or effecting such a response or improvement in function or resilience within a component of the nervous system. For example, examples of producing a neurosalutary effect would include stimulating axonal outgrowth after injury to a neuron; rendering a neuron resistant to apoptosis; rendering a neuron resistant to a toxic compound such as β-amyloid, ammonia, or other neurotoxins; reversing age-related neuronal atrophy or loss of function; or reversing age-related loss of cholinergic innervation.

Any agent that activates the growth pathway of CNS neurons is suitable for use in the methods of the present invention. Some preferred agents include but are not limited to inosine, mannose, gulose, or glucose-6-phosphate, as described in Li et. al., 2003, J. Neuroscience 23(21):7830-7838; Chen Et al., 2002, Proc. Natl. Acad. Sci. U.S.A, 99:1931-1936; and Benowitz et al., 1998 J. Biol. Chem. 273:29626-29634, which are herein incorporated by reference in their entirety. TGF-β, and oncomodulin as described in Yin et al., 2003, J. Neurosci., 23: 2284-2293, are also preferred agents. In addition, polypeptide growth factors such as BDNF, NGF, NT-3, CNTF, LIF, and GDNF can be used. In one embodiment the methods of the present invention further comprise contacting CNS neurons with a cAMP modulator that increases the concentration of intracellular cAMP. For example, the ability of mature rat retinal ganglionic cells to respond to mannose requires elevated cAMP (Li et. al., 2003, J. Neuroscience 23(21):7830-7838).

The ability of an agent to activate the growth pathway of CNS neurons in a subject may be assessed using any of a variety of known procedures and assays. For example, the ability of an agent to re-establish neural connectivity and/or function after an CNS injury, may be determined histologically (either by slicing neuronal tissue and looking at neuronal branching, or by showing cytoplasmic transport of dyes). Agents may also be assessed by monitoring the ability of the agent to fully or partially restore the electroretinogram after damage to the neural retina or optic nerve; or to fully or partially restore a pupillary response to light in the damaged eye.

Other tests that may be used to determine the ability of an agent to produce a neurosalutary effect in a subject include standard tests of neurological function in human subjects or in animal models of spinal injury (such as standard reflex testing, urologic tests, urodynamic testing, tests for deep and superficial pain appreciation, propnoceptive placing of the hind limbs, ambulation, and evoked potential testing). In addition, nerve impulse conduction can be measured in a subject, such as by measuring conduct action potentials, as an indication of the production of a neurosalutary effect.

Animal models suitable for use in the assays of the present invention include the rat model of partial transaction (described in Weidner et al., 2001). This animal model tests how well a compound can enhance the survival and sprouting of the intact remaining fragment of an almost fully-transected cord. Accordingly, after administration of a candidate agent these animals may be evaluated for recovery of a certain function, such as how well the rats may manipulate food pellets with their forearms (to which the relevant cord had been cut 97%).

Another animal model suitable for use in the assays of the present invention includes the rat model of stroke (described in Kawamata et al., 1997). This paper describes in detail various tests that may be used to assess sensor motor function in the limbs as well as vestibulomotor function after an injury. Administration to these animals of the compounds of the invention can be used to assess whether a given compound, route of administration, or dosage provides a neurosalutary effect, such as increasing the level of function, or increasing the rate of regaining function or the degree of retention of function in the test animals.

Standard neurological evaluations used to assess progress in human patients after a stroke may also be used to evaluate the ability of an agent to produce a neurosalutary effect in a subject. Such standard neurological evaluations are routine in the medical arts, and are described in, for example, “Guide to Clinical Neurobiology” Edited by Mohr and Gautier (Churchill Livingstone Inc. 1995).

### Pharmaceutically Acceptable Formulations

The agents of the present invention can be contained in pharmaceutically acceptable formulations. Such pharmaceutically acceptable formulation may include a pharmaceutically acceptable carrier(s) and/or excipient(s). As used herein, “pharmaceutically acceptable carrier” includes any and all solvents, dispersion media, coatings, antibacterial and anti fungal agents, isotonic and absorption delaying agents, and the like that are physiologically compatible. For example, the carrier can be suitable for injection into the cerebrospinal fluid. Excipients include pharmaceutically acceptable stabilizers. The present invention pertains to any pharmaceutically acceptable formulations, including synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes.

In one embodiment, the pharmaceutically acceptable formulations comprise a polymeric matrix. The terms “polymer” or “polymeric” are art-recognized and include a structural framework comprised of repeating monomer units which is capable of delivering a hexose derivative such that treatment of a targeted condition, such as a neurological disorder, occurs. The terms also include co-polymers and homopolymers such as synthetic or naturally occurring. Linear polymers, branched polymers, and cross-linked polymers are also meant to be included.

For example, polymeric materials suitable for forming the pharmaceutically acceptable formulation employed in the present invention, include naturally derived polymers such as albumin, alginate, cellulose derivatives, collagen, fibrin, gelatin, and polysaccharides, as well as synthetic polymers such as polyesters (PLA, PLGA), polyethylene glycol, poloxomers, polyanhydrides, and pluronics. These polymers are biocompatible with the nervous system, including the central nervous system, they are biodegradable within the central nervous system without producing any toxic byproducts of degradation, and they possess the ability to modify the manner and duration of the active compound release by manipulating the polymer's kinetic characteristics. As used herein, the term “biodegradable” means that the polymer will degrade over time by the action of enzymes, by hydrolytic action and/or by other similar mechanisms in the body of the subject. As used herein, the term “biocompatible” means that the polymer is compatible with a living tissue or a living organism by not being toxic or injurious and by not causing an immunological rejection. Polymers can be prepared using methods known in the art.

The polymeric formulations can be formed by dispersion of the active compound within liquefied polymer, as described in U.S. Pat. No. 4,883,666, the teachings of which are incorporated herein by reference or by such methods as bulk polymerization, interfacial polymerization, solution polymerization and ring polymerization as described in Odian G., Principles of Polymerization and ring opening polymerization, 2nd ed., John Wiley & Sons, New York, 1981, the contents of which are incorporated herein by reference. The properties and characteristics of the formulations are controlled by varying such parameters as the reaction temperature, concentrations of polymer and the active compound, the types of solvent used, and reaction times.

The active therapeutic compound can be encapsulated in one or more pharmaceutically acceptable polymers, to form a microcapsule, microsphere, or microparticle, terms used herein interchangeably. Microcapsules, microspheres, and microparticles are conventionally free-flowing powders consisting of spherical particles of 2 millimeters or less in diameter, usually 500 microns or less in diameter. Particles less than 1 micron are conventionally referred to as nanocapsules, nanoparticles or nanospheres. For the most part, the difference between a microcapsule and a nanocapsule, a microsphere and a nanosphere, or microparticle and nanoparticle is size; generally there is little, if any, difference between the internal structure of the two. In one aspect of the present invention, the mean average diameter is less than about 45 μm, preferably less than 20 μm, and more preferably between about 0.1 and 10 μm.

In another embodiment, the pharmaceutically acceptable formulations comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidylcholine, dioleoylphosphatidylcholine, dipalmitoylphosphatidylglycerol, and dioleoylphosphatidylglycerol.

In preparing lipid-based vesicles containing an active compound such variables as the efficiency of active compound encapsulation, liability of the active compound, homogeneity and size of the resulting population of vesicles, active compound-to-lipid ratio, permeability, instability of the preparation, and pharmaceutical acceptability of the formulation should be considered.

Prior to introduction, the formulations can be sterilized, by any of the numerous available techniques of the art, such as with gamma radiation or electron beam sterilization.

Ophthalmic products for topical use may be packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquarternium-1, or other agents known to those skilled in the art. Such preservatives are typically employed at a level of from 0.001 to 1.0% weight/volume (“% w/v”). Such preparations may be packaged in dropper bottles or tubes suitable for safe administration to the eye, along with instructions for use.

### Administration of the Pharmaceutically Acceptable Formulations to a Patient

When the agents are delivered to a patient, they can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated. Agents can also be delivered using viral vectors, which are well known to those skilled in the art.

The compounds are administered such as the agents come into contact with a subject's nervous system. The preferred mode of administration can vary depending upon the particular agent chosen.

Both local and systemic administration are contemplated by the invention. Desirable features of local administration include achieving effective local concentrations of the active compound as well as avoiding adverse side effects from systemic administration of the active compound. In one embodiment, the active agents are administered by introduction into the cerebrospinal fluid of the subject. In certain aspects of the invention, the active compound is introduced into a cerebral ventricle, the lumbar area, or the cistema magna. In another aspect, the active compound is introduced locally, such as into the site of nerve or cord injury, into a site of pain or neural degeneration, or intraocularly to contact neuroretinal cells.

The pharmaceutically acceptable formulations can be suspended in aqueous vehicles and introduced through conventional hypodermic needles or using infusion pumps.

In one embodiment, the active compound formulation described herein is administered to the subject in the period from the time of, for example, an injury to the CNS up to about 100 hours after the injury has occurred, for example within 24, 12, or 6 hours from the time of injury.

In another embodiment of the invention, the active compound formulation is administered into a subject intrathecally. As used herein, the term “intrathecal administration” is intended to include delivering an active compound formulation directly into the cerebrospinal fluid of a subject, by techniques including lateral cerebroventricular injection through a burrhole or cistemal or lumbar puncture or the like (described in Lazorthes et al., 1991, and Ommaya A. K., 1984, the contents of which are incorporated herein by reference). The term “lumbar region” is intended to include the area between the third and fourth lumbar (lower back) vertebrae. The term “cistema magna” is intended to include the area where the skull ends and the spinal cord begins at the back of the head. The ten-n “cerebral ventricle” is intended to include the cavities in the brain that are continuous with the central canal of the spinal cord. Administration of an active compound to any of the above mentioned sites can be achieved by direct injection of the active compound formulation or by the use of infusion pumps. Implantable or external pumps and catheter may be used.

For injection, the active compound formulation of the invention can be formulated in liquid solutions, preferably in physiologically compatible buffers such as Hank's solution or Ringer's solution. In addition, the active compound formulation may be formulated in solid form and re-dissolved or suspended immediately prior to use. Lyophilized forms are also included. The injection can be, for example, in the form of a bolus injection or continuous infusion (such as using infusion pumps) of the active compound formulation.

In one embodiment of the invention, the active compound formulation is administered by lateral cerebroventricular injection into the brain of a subject, preferably within 100 hours of when an injury (resulting in a condition characterized by aberrant axonal outgrowth of central nervous system neurons) occurs (such as within 6, 12, or 24 hours of the time of the injury). The injection can be made, for example, through a burr hole made in the subject's skull. In another embodiment, the formulation is administered through a surgically inserted shunt into the cerebral ventricle of a subject, preferably within 100 hours of when an injury occurs (such as within 6, 12 or 24 hours of the time of the injury). For example, the injection can be made into the lateral ventricles, which are larger, even though injection into the third and fourth smaller ventricles can also be made. In yet another embodiment, the active compound formulation is administered by injection into the cistema magna, or lumbar area of a subject, preferably within 100 hours of when an injury occurs (such as within 6, 12, or 24 hours of the time of the injury).

An additional means of administration to intracranial tissue involves application of compounds of the invention to the olfactory epithelium, with subsequent transmission to the olfactory bulb and transport to more proximal portions of the brain. Such administration can be by nebulized or aerosolized preparations.

In another embodiment of the invention, the active compound formulation is administered to a subject at the site of injury, preferably within 100 hours of when an injury occurs (such as within 6, 12, or 24 hours of the time of the injury).

In a further embodiment, ophthalmic compositions of the present invention are used to prevent or reduce damage to retinal and optic nerve head tissues, as well as to enhance functional recovery after damage to ocular tissues. Ophthalmic conditions that may be treated include, but are not limited to, retinopathies (including diabetic retinopathy and retrolental fibroplasia), macular degeneration, ocular ischemia, glaucoma. Other conditions to be treated with the methods of the invention include damage associated with injuries to ophthalmic tissues, such as ischemia reperfusion injuries, photochemical injuries, and injuries associated with ocular surgery, particularly injuries to the retina or optic nerve head by exposure to light or surgical instruments. The ophthalmic compositions may also be used as an adjunct to ophthalmic surgery, such as by vitreal or subconjunctival injection following ophthalmic surgery. The compounds may be used for acute treatment of temporary conditions, or may be administered chronically, especially in the case of degenerative disease. The ophthalmic compositions may also be used prophylactically, especially prior to ocular surgery or noninvasive ophthalmic procedures or other types of surgery.

### Duration and Levels of Administration

In a preferred embodiment of the method of the invention, the active compound is administered to a subject for an extended period of time to produce optimum axonal outgrowth. Sustained contact with the active compound can be achieved by, for example, repeated administration of the active compound over a period of time, such as one week, several weeks, one month or longer. More preferably, the pharmaceutically acceptable formulation used to administer the active compound provides sustained delivery, such as “slow release” of the active compound to a subject. For example, the formulation may deliver the active compound for at least one, two, three, or four weeks after the pharmaceutically acceptable formulation is administered to the subject. Preferably, a subject to be treated in accordance with the present invention is treated with the active compound for at least 30 days (either by repeated administration or by use of a sustained delivery system, or both).

As used herein, the term “sustained delivery” is intended to include continual delivery of the active compound in vivo over a period of time following administration, preferably at least several days, a week, several weeks, one month or longer. Sustained delivery of the active compound can be demonstrated by, for example, the continued therapeutic effect of the active compound over time (such as sustained delivery of the agents can be demonstrated by continued axonal growth in CNS neurons in a subject). Alternatively, sustained delivery of the active compound may be demonstrated by detecting the presence of the active compounds in vivo over time.

Preferred approaches for sustained delivery include use of a polymeric capsule, a minipump to deliver the formulation, a biodegradable implant, or implanted transgenic autologous cells (as described in U.S. Pat. No. 6,214,622). Implantable infusion pump systems (such as Infusaid; see such as Zierski, J. et al, 1988; Kanoff, R. B., 1994) and osmotic pumps (sold by Alza Corporation) are available in the art. Another mode of administration is via an implantable, externally programmable infusion pump. Suitable infusion pump systems and reservoir systems are also described in U.S. Pat. No. 5,368,562 by Blomquist and U.S. Pat. No. 4,731,058 by Doan, developed by Pharmacia Deltec Inc.

It is to be noted that dosage values may vary with the severity of the condition to be alleviated. It is to be further understood that for any particular subject, specific dosage regimens should be adjusted over time according to the individual need and the professional judgment of the person administering or supervising the administration of the active compound and that dosage ranges set forth herein are exemplary only and are not intended to limit the scope or practice of the claimed invention.

The amount of agent administered to the individual will depend on the characteristics of the individual, such as general health, age, sex, body weight and tolerance to drugs as well as the degree, severity and type of rejection. The skilled artisan will be able to determine appropriate dosages depending on these and other factors. Typically, an effective amount can range from about 0.1 mg per day to about 100 mg per day for an adult. Preferably, the dosage ranges from about 1 mg per day to about 100 mg per day.

Antibodies and antigen-binding fragments thereof, particularly human, humanized and chimeric antibodies and antigen-binding fragments can often be administered less frequently than other types of therapeutics. For example, an effective amount of such an antibody can range from about 0.01 mg/kg to about 5 or 10 mg/kg administered daily, weekly, biweekly, monthly or less frequently.

### In Vitro Treatment of Neurons

Neurons derived from the central or peripheral nervous system can be contacted with the agents ex vivo to modulate axonal outgrowth in vitro. Accordingly, neurons can be isolated from a subject and grown in vitro, using techniques well known in the art, and then treated in accordance with the present invention to modulate axonal outgrowth. Briefly, a neuronal culture can be obtained by allowing neurons to migrate out of fragments of neural tissue adhering to a suitable substrate (such as a culture dish) or by disaggregating the tissue, such as mechanically or enzymatically, to produce a suspension of neurons. For example, the enzymes trypsin, collagenase, elastase, hyaluronidase, DNase, pronase, dispase, or various combinations thereof can be used. Methods for isolating neuronal tissue and the disaggregation of tissue to obtain isolated cells are described in Freshney, Culture of Animal Cells, A Manual of Basic Technique, Third Ed., 1994, the contents of which are incorporated herein by reference.

Such cells can be subsequently contacted with the agents (alone or in combination with a cAMP modulator) in amounts and for a duration of time as described above. Once modulation of axonal outgrowth has been achieved in the neurons, these cells can be re-administered to the subject, such as by implantation.

### Treatment of Neurological Disorders

Elements of the nervous system subject to disorders which may be effectively treated with the compounds and methods of the invention include the central, somatic, autonomic, sympathetic and parasympathetic components of the nervous system, neurosensory tissues within the eye, ear, nose, mouth or other organs, as well as glial tissues associated with neuronal cells and structures. Neurological disorders may be caused by an injury to a neuron, such as a mechanical injury or an injury due to a toxic compound, by the abnormal growth or development of a neuron, or by the misregulation, such as downregulation, of an activity of a neuron. Neurological disorders can detrimentally affect nervous system functions such as the sensory function (the ability to sense changes within the body and the outside environment); the integrative function (the ability to interpret the changes); and the motor function (the ability to respond to the interpretation by initiating an action such as a muscular contraction or glandular secretion).

Examples of neurological disorders include traumatic or toxic injuries to peripheral or cranial nerves, spinal cord or to the brain, cranial nerves, traumatic brain injury, stroke, cerebral aneurism, and spinal cord injury. Other neurological disorders include cognitive and neurodegenerative disorders such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, hereditary motor and sensory neuropathy (Charcot-Marie-Tooth disease), diabetic neuropathy, progressive supranuclear palsy, epilepsy, and Jakob-Creutzfieldt disease. Autonomic function disorders include hypertension and sleep disorders.

Also to be treated with compounds and methods of the invention are neuropsychiatric disorders such as depression, schizophrenia, schizoaffective disorder, Korsakoff s psychosis, mania, anxiety disorders, or phobic disorders, learning or memory disorders (such as amnesia and age-related memory loss), attention deficit disorder, dysthymic disorder, major depressive disorder, mania, obsessive-compulsive disorder, psychoactive substance use disorders, anxiety, phobias, panic disorder, bipolar affective disorder, psychogenic pain syndromes, and eating disorders. Other examples of neurological disorders include injuries to the nervous system due to an infectious disease (such as meningitis, high fevers of various etiologies, HIV, syphilis, or post-polio syndrome) and injuries to the nervous system due to electricity (including contact with electricity or lightning, and complications from electro-convulsive psychiatric therapy). The developing brain is a target for neurotoxicity in the developing central nervous system through many stages of pregnancy as well as during infancy and early childhood, and the methods of the invention may be utilized in preventing or treating neurological deficits in embryos or fetuses in utero, in premature infants, or in children with need of such treatment, including those with neurological birth defects. Further neurological disorders include, for example, those listed in Harrison's Principles of Internal Medicine (Braunwald et al., McGraw-Hill, 2001) and in the American Psychiatric Association's Diagnostic and Statistical Manual of Mental Disorders DSM-IV (American Psychiatric Press, 2000) both incorporated herein by reference in their entirety. Neurological disorders associated with ophthalmic conditions include retina and optic nerve damage, glaucoma and age related macular degeneration.

As used herein, the term “stroke” is art recognized and is intended to include sudden diminution or loss of consciousness, sensation, and voluntary motion caused by rupture or obstruction (for example, by a blood clot) of an artery of the brain.

As used herein, “Traumatic brain injury” is art recognized and is intended to include the condition in which, a traumatic blow to the head causes damage to the brain or connecting spinal cord, often without penetrating the skull. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLES

### Example I

### NgR Mediates Axon Regeneration in Mature CNS

The optic nerve is a classic model for understanding regenerative failure or success in the mature mammalian CNS (Aguayo et al., 1991; Ramon y Cajal, 1991). Axons that are injured in the mature rat optic nerve cannot grow back into the myelin-rich environment distal to the injury site. In addition, if axonal damage occurs close to the eye, retinal ganglion cells (RGCs) undergo apoptosis after several days (Berkelaar et al., 1994). Several intraocular manipulations, including injuring the lens (Leon et al., 2000; Fischer et al., 2000, 2001), injecting the pro-inflammatory agent zymosan (Yin et al., 2003), or inserting a peripheral nerve fragment (Berry et al., 1996), partially reverse this situation and allow many RGCs to survive injury and regenerate lengthy axons into the optic nerve; these effects appear to be mediated via macrophage-derived factors (Yin et al., 2003) acting in concert with a carbohydrate that is constitutively present in the eye (Li et al., 2003). The partial regeneration that occurs under these conditions provides a sensitized background on which to investigate the significance of NgR in CNS regeneration. This was done here by transfecting RGCs with adeno-associated viruses (AAV) carrying a gene for either the wild-type NgR or for NgR^{DN}.

### Materials and Methods

### Viral Transfections.

cDNAs encoding either wild-type NgR (Fournier et al., 2001) or a C-terminal truncated, dominant-negative variant of NgR that retains the ligand binding domain does not associate with its co-receptor (Domeniconi et al., 2002; Wang et al., 2002b), were inserted into the AAV-MCS2-IGFP plasmid, described on the website of the Harvard Gene Therapy Initiative ( ). Gene expression was driven by a CMV promoter. Constructs expressed enhanced green fluorescent protein (GFP) from an internal ribosome entry site. NgR constructs obtained an HA epitope tag, as described (Wang et al., 2002a). Controls were transfected with viruses expressing GFP alone. Virus production was carried out at the Harvard Gene Therapy Initiative Core Facility. To transfect RGCs, female Sprague-Dawley rats (160-180 g) were anesthetized with Ketamine-Xylazine and the back of the eye was exposed intraorbitally. After withdrawing 10 μl of fluid from the eye, ^{˜}10¹⁰ AAV particles in 10 μl phosphate-buffered saline (PBS) were injected into the vitreous body using a micropipette, with care taken to avoid injuring the lens (Fischer et al., 2000). Injections were done 3 weeks prior to optic nerve surgery to maximize levels of transgene expression at the onset of axon regeneration (Cheng et al., 2002).

### Optic Nerve Surgery and Lens Injury.

Animals were re-anesthetized using Ketamine-Xylazine, immobilized in a stereotaxic apparatus, and the left optic nerve was surgically exposed intraorbitally. After opening the meninges longitudinally, the optic nerve was crushed 2 mm from the orbit by applying pressure with jewelers' forceps under a dissecting microscope for 10 sec. Lens injury was accomplished by puncturing the lens capsule with a microcapillary through a posterior approach (Fischer et al., 2000). Lens injury leads to macrophage activation, and factors secreted from activated macrophages stimulate RGCs to regenerate their axons (Yin et al., 2003). Controls sustained nerve injury but no lens damage. Nerve injury was verified by the appearance of a clearing at the crush site; the vascular integrity of the retina was verified by fundoscopic examination.

### Retinal Explants.

Explants of viral-transfected retinas were prepared 4 days after crushing the optic nerve and either injuring the lens or performing sham surgery. Animals were euthanized and their retinas were dissected out, cut into 8 radial pieces, and cultured in DMEM-B27 (Invitrogen) on a laminin-poly-D-lysine substrate (Bahr et al., 1988) with or without myelin, prepared as described (Wang et al., 2002b). Two days later, the number of axons growing ≧50 μm beyond the margin of each explant was counted with the aid of an inverted phase-contrast microscope (Axiovert, Zeiss) and a calibrated ocular micrometer at a magnification of ×200. In cases with strong regeneration, some fiber fasciculation was observed, and these were counted as single axons. Results from individual explants were averaged within each treatment group and between-group differences were evaluated with Student's t-test. To evaluate growth on myelin, we calculated the ratio of axons growing >500 μm to total axons ≧50 μm in TUJ1-immunostained explants. This was done to account for the variability in adhesion and outgrowth of explants grown on the mixed myelin-laminin substrate, and to visualize axons against a particulate background. Results were averaged from 6 explants per retina and 4-5 retinas per condition.

### Histology: Retinal Explants.

After 2 days in culture, retinas were fixed in 4% paraformaldehyde in PBS, treated with methanol for 10 min, blocking solution containing 10% serum from the same species as the secondary antibody for 1 hour (RT), and then incubated overnight (4° C.) with antibodies against either GFP (prepared in rabbit: Molecular Probes, Eugene, Oreg., 1:1000); βIII tubulin (mouse monoclonal antibody TUJ1, Babco, Richmond, Calif., 1:500), or the HA epitope tag (mouse monoclonal antibody, Molecular Probes, 1:100) fused to NgR. Primary antibodies were prepared in Tris-buffered saline (TBS) containing 2× physiological saline, 5% serum, 2% BSA, and 0.1% Tween-20. Following 3 rinses in TBS, sections were incubated with fluorescently tagged secondary antibodies, i.e., AlexaFluor 488-conjugated goat antibody to rabbit IgG or AlexaFluor 594-conjugated goat antibody to mouse IgG (1:500, 2 hours, RT), rinsed, and covered.

### Optic Nerve and Retinal Cross-Sections.

Two weeks after nerve surgery, animals were euthanized with an overdose of anesthesia and perfused with PBS followed by 4% paraformaldehyde in PBS. Optic nerves with retinas attached were dissected and prepared for longitudinal sectioning as described (Yin et al., 2003). Sections were stained to visualize either GAP-43 (primary antibody prepared in sheep (Benowitz et al., 1988); 1:1000, followed by a fluorescent-tagged donkey anti-sheep IgG), or GFP, as above. Retinal cross-sections were stained to visualize either GFP or βIII tubulin (as above), or NgR. The latter was visualized using a primary antibody made in goat to the N-terminus of NgR (1:10, Santa Cruz), followed by a fluorescent secondary antibody to goat IgG made in donkey (1:500).

### Axon Regeneration: Quantitation.

Regeneration was quantified as described (Leon et al., 2000; Yin et al, 2003). In brief, under 400× magnification, we counted the number of GAP-43 positive axons extending >500 μm and >1 mm from the injury site in 4 sections per case, normalized these numbers to the cross-sectional width of the optic nerve, and used these data to calculate the total numbers of regenerating axons in each animal (Leon et al., 2000; Yin et al, 2003). The significance of inter-group differences were evaluated by Student's t-tests.

### Cell Survival

Cross-sections through the center of the retina were double-stained with antibodies to GFP and βIII tubulin as described above. The numbers of βIII tubulin-positive cells per section were counted in 4-6 sections per case, averaged for each case, and then averaged across all similarly treated animals to obtain group means and standard errors.

### Results

To investigate the role of NgR in vivo, we injected mature rats intravitreally with AAV (serotype 2) carrying a plasmid expressing either the wild-type Nogo receptor (NgR^{WT})(Fournier et al., 2001) or a truncated, dominant-negative variant of NgR (NgR^{DN})(Domeniconi et al., 2002; Wang et al., 2002b) from a CMV promoter, along with enhanced green fluorescent protein (GFP) from an internal ribosome entry site (AAV-NgR^{WT}-IGFP and AAV-NgR^{DN}-IGFP, respectively). Controls were transfected with viruses expressing GFP alone (AAV-GFP). When examined 3 weeks later, the GFP reporter was detected in >75% of all RGCs, in agreement with prior studies using a similar virus (Cheng et al., 2002; Martin et al., 2002). GFP-labeled cells were localized almost exclusively within the ganglion cell layer in cells that are immunopositive for βIII tubulin. Within the retina, this tubulin isoform is expressed only in RGCs (Cui et al., 2003: Yin et al., 2003), which we verified by showing a complete overlap of βIII tubulin immunostaining with Fluorogold labeling in RGCs after injecting the latter into the superior colliculus. The specificity of transfection to RGCs presumably reflects a combination of the neural-selectivity of AAV2 (Bartlett et al., 1998) and the ready access of intravitreal viral particles of RGC axons and somata.

NgR immunostaining was modest or weak in controls transfected with AAV-GFP, but was strong in retinas transfected with AAV-NgR^{WT}-IGFP. Thus, in transfected cells, levels of transgene expression exceed those of the endogenous protein. Three weeks after transfections, animals were re-anesthetized and the left optic nerve was crushed 2 mm from the back of the eye; in half of these animals, the lens was damaged to activate macrophages and promote regeneration (Fischer et al., 2000; Leon et al., 2000; Yin et al., 2003); the remaining animals received no further surgery.

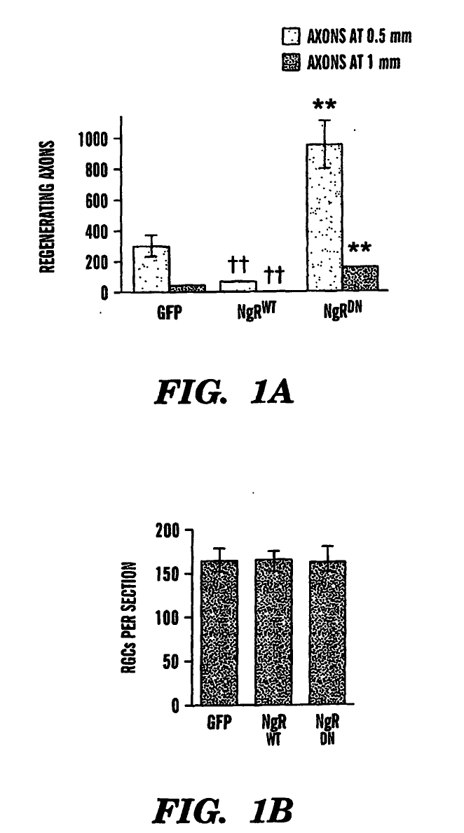

Regeneration was investigated 2 weeks after optic nerve injury; prior work has shown that damaged axons have begun to grow back into the distal optic nerve by this time provided macrophages have been activated intravitreally (Leon et al., 2000). Regenerating axons are readily distinguished by staining with antibodies to GAP-43. GAP-43 is normally undetectable in the mature optic nerve but is strongly upregulated in RGC axons undergoing regeneration (Schaden et al., 1994; Berry et al., 1996; Leon et al., 2000). The origin of the GAP-43 positive axons in RGCs has been shown previously by anterograde labeling and double-immunostaining (Leon et al., 2000). Controls transfected with AAV-GFP (n=8) showed a moderate number of GAP-43-positive axons distal to the injury site, in numbers comparable to those reported in similarly treated animals without viral transfections (FIG. 1A; Leon et al., 2000).

Two weeks after nerve crush and lens injury, animals overexpressing NgR^{WT} showed 76% fewer axons regenerating ≧0.5 mm from the injury site than controls (n=9, p<0.01), and 96% fewer axons extending ≧1 mm (p<0.01). Many NgR^{WT}-containing axons retracted from the lesion site towards the optic nerve head, reflecting the sensitivity of these axons to myelin; this phenomenon was never observed in animals expressing GFP alone or NgR^{DN}.

In striking contrast, expression of NgR^{DN} enhanced axon regeneration greatly. Two weeks after nerve crush and lens injury, animals expressing NgR^{DN} (n=5) extended approximately 3 times more axons >1 mm beyond the injury site than controls expressing GFP alone, and 75 times more axons than animals expressing NR^{WT} (FIG. 1A). In general, although GFP could be visualized in many axons proximal to the injury site, fewer than half of the axons that extended beyond this point exhibited GFP immunofluorescence, presumably due to decreasing concentrations of the cytoplasmic reporter protein far from RGC somata. However, the longest regenerating axons frequently exhibited GFP staining, which suggests that they may have arisen from RGCs that express abundant NgR^{DN}. This co-localization further confirms the origin of GAP-43 immunopositive axons in RGCs. Diminished transgene expression combined with declining RGC viability after longer survival times probably limits the amount of regeneration that can be obtained under the present conditions, and further research will be required to determine whether overcoming these problems will enable growth-activated, NgR^{DN}-expressing RGCs to extend axons back to their central targets.

In the absence of lens injury, NgR^{DN} expression did not enable RGCs to regenerate their axons into the distal optic nerve. Quantitatively, no axons were counted at 0.5 mm in any animal without lens injury irrespective of which transgene was expressed.

To investigate whether the effects of the 3 transgenes on axon regeneration might reflect differences in cell survival, we counted TUJ1-positive cells in retinal cross-sections 2 weeks after nerve crush and lens injury. Transgene expression had no measurable effect on cell survival (FIG. 1B).

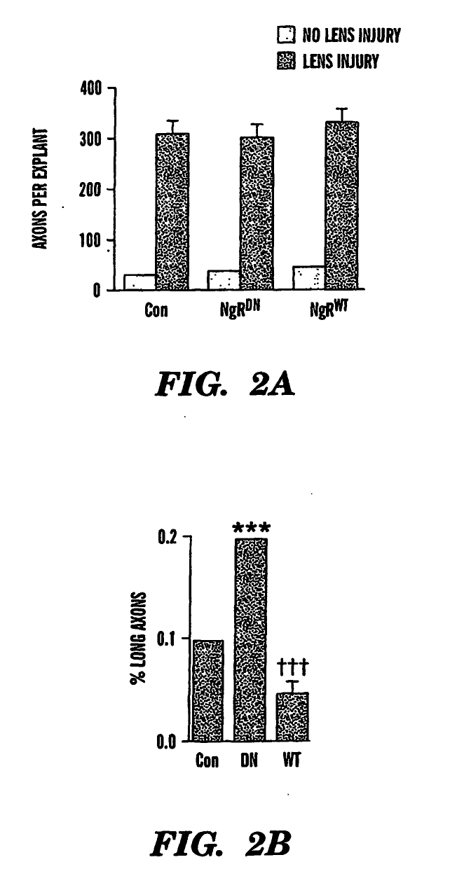

To investigate whether altering NgR levels or function might affect RGCs' intrinsic ability to extend axons, we investigated outgrowth on a more permissive substrate. As before, we transfected RGCs in vivo with either AAV-NgR^{WT}-IGFP or AAV-NgR^{DN}-IGFP, then performed optic nerve surgery combined with lens injury or sham intraocular surgery 3 weeks later. After 4 days, a time at which axotomized RGCs stimulated by macrophage-derived factors go into a growth state (Fischer et al., 2000), we explanted wedges of retinas onto a poly-L-lysine-laminin (PLL) substrate. Little outgrowth was seen in explants not exposed to growth factors in vivo irrespective of transgene expression (FIG. 2A). It should be noted that axotomized RGCs do not show signs of apoptosis at this time point (Berkelaar et al., 1994). Retinas primed to grow as a result of lens injury in vivo showed strong outgrowth regardless of which transgene was expressed (FIG. 2A). There was strong outgrowth from RGCs expressing NgR^{WT}, while minimal outgrowth from a growth-activated retina expressing NgR^{DN}.

As expected, the effects of transgene expression became apparent when explants were plated on a substrate containing myelin (FIG. 2B). NgR^{WT} overexpression decreased the percentage of axons growing >500 μm on a mixed myelin-laminin substrate by approximately 50% relative to controls, whereas expression of NgR^{DN} doubled the percentage of long axons (p<0.001 in both cases).

### Discussion

The results of this study show that NgR plays a major role in limiting axon regeneration in the mature optic nerve; however, extensive regeneration requires activation of neurons' intrinsic growth state in addition to suppression of NgR activity. Our results also demonstrate that AAV-mediated transfection provides a highly effective means of altering either the levels of functioning of gene products important for axon regeneration in CNS neurons.

The critical role of NgR for optic nerve regeneration is evident from the dramatic enhancement of axon growth that occurs when growth-sensitized RGCs express a dominant-negative form of NgR, and conversely, from the near-complete failure of sensitized RGCs to regenerate their axons when overexpressing wild-type NgR. In mature mice, a null mutation of the NgR gene does not enhance regeneration of the corticospinal tract (CST), but does increase sprouting of essential descending serotonergic projections after spinal cord injury (Kim et al., 2003a). Based upon the present study, we would propose that the contrasting results seen in CST vs. serotonergic axons after NgR deletion may reflect intrinsic differences in the growth state of cortical pyramidal cells vs. raphe neurons, and that activation of the former with appropriate trophic factors could lead to a stronger CST phenotype.

Alterations of NgR functioning (or levels) and activation of the axonal growth program are largely independent of one another. As shown in the explant studies, altering NgR functioning or levels did not affect neurons' ability to extend axons on a permissive substrate, and activating RGCs' intrinsic growth state still left axons partially responsive to the effects of myelin proteins. Activation of RGCs' growth program by macrophage-derived factors greatly increases the expression of GAP-43 (Yin et al., 2003) and other regeneration-associated genes, but does not appreciably alter mRNA levels of NgR or p75, a NgR co-receptor (D. Fischer and L. Benowitz, unpublished gene profiling results). Inhibition of RhoA, an essential downstream mediator of NgR functioning, allows for limited axon regeneration when an ADP ribosyl transferase is delivered at the site of optic nerve injury (Lehmann et al., 1999).

AAV-mediated transfection of growth-sensitized RGCs represents a general approach for investigating the role of various gene products in axon regeneration. By this method, one can readily obtain precise temporal and spatial control of gene expression without the expense, time delays, and possible developmental problems inherent in transgenic technology. The specificity and efficiency of RGC transfection by AAV found here has also been demonstrated in other studies (Cheng et al., 2002; Martin et al., 2002).

The clinical implications of this work are clear: extensive axon regeneration is not attainable in the mature CNS by overcoming inhibitory signals alone, but requires that neurons' intrinsic growth state be activated at the same time (Schnell et al., 1994; Cheng et al., 1996; Guest et al., 1997).

### Example II

### RhoA Inactivation Combined with Lens Injury Results in High Levels of Axon Regeneration

### Materials And Methods

### Induction of Axon Regeneration

Adult female Sprague Dawley rats, 220-250 gm, were anesthetized by intraperitoneal injection of ketamine (60-80 mg/kg) and xylazine (10-15 mg/kg), and a 1-1.5 cm incision was made in the skin above the right orbit. The optic nerve was surgically exposed under an operating microscope, the epineurium was opened longitudinally, and the nerve was crushed 0.5 mm behind the eye for 10 sec using jeweler's forceps, avoiding injury to the ophthalmic artery. Nerve injury was verified by the appearance of a clearing at the crush site; the vascular integrity of the retina was verified by fundoscopic examination. Lens injury was induced through a retrolenticular approach, puncturing the lens capsule with the narrow tip of a microcapillary tube; inflammation was enhanced by injecting 10 μl of PBS intravitreally after retrieving the same volume from the anterior chamber of the eye (Fischer et al., 2000). Controls received PBS injections only. All surgical procedures were approved by the Institutional Animal Care and Use Committee of Children's Hospital.

### Retinal Explants

Rats were killed, and their retinas were dissected 0-7 d after crushing the optic nerve and either injuring the lens or performing sham intraocular surgery (n=5 animals per group). Additional controls received no treatment (n=5) or lens injury without nerve crush (n=5). Retinas were cut into eight radial pieces, which were cultured in astrocyte-microglia growth medium (PromoCeli, Heidelberg, Germany) in laminin-poly-L-lysine-coated dishes (Bahr et al, 1988). In some cases, we coated culture plates with myelin (courtesy of Dr. Zhigang He, Children's Hospital, Boston, Mass.), as described (Wang et al., 2002a). The number of axons extending ≧50 μm from each explant was counted after 24 and 48 hr using inverted phase-contrast optics (200×; Axiovert; Zeiss, Thornwood, N.Y.) and a calibrated ocular micrometer. In cases with strong regeneration, some fiber fasciculation was observed, and these were counted as one axon. Results from individual explants were averaged within each experimental group, and intergroup differences were evaluated by Student's 't test. Growth velocities were estimated after at least five axons had extended from the edge of the explant. The lengths of these five axons were measured at 4, 6, 12, 18, 24, 36, and 48 hr.

### Immunohistochemistry

Animals were killed with a lethal overdose of anesthesia and perfused through the heart with cold saline plus heparin, followed by 4% paraformaldehyde. Eyes with optic nerves segments attached were dissected from connective tissue, postfixed overnight, transferred to 30% sucrose overnight (4° C.), and frozen. Frozen sections were cut longitudinally on a cryostat, thaw-mounted onto coated glass slides (Superfrost plus; Fisher Scientific, Pittsburgh, Pa.), and stored at −20° C. until additional use. To visualize RGCs in double-labeling experiments, we used the monoclonal mouse TUJ1 antibody (Babco, Richmond, Calif.) at a dilution of 1:500. Secondary antibodies included a cyanine 3-conjugated anti-rabbit 1 gG antibody (1:600; Jackson ImmunoResearch, West Grove, Pa.) and anti-mouse IgG conjugated to Alexa Flour 488 (1:500; Molecular Probes). Fluorescent sections were covered using Vectashield mounting medium (Vector Laboratories) and analyzed under a fluorescent microscope.

### Visualization of RhoA Activation by Rho-Binding Domain-Glutathione S-Transferase Staining

The Rho-binding domain (RBD) of the protein rhotekin binds selectively to the active (GTP-bound) form of RhoA and can be used as a reagent to visualize RhoA-GTP in cell homegenates or in situ (Dubreuil et al., 2002). Bacteria expressing a glutathione S-transferase (GST)-RBD fusion protein in a pGEX vector (a gift from John Collard, Division of Cell Biology, Netherlands Cancer Institute, Amsterdam, The Netherlands) were grown in L-broth with 100 μl/ml ampicillin. Overnight cultures were diluted 1:10 into 1000 ml of L-broth and incubated in a shaking bacterial incubator at 37° C. for 1 hr. Isopropl-β-D-thiogalactopyranoside was then added to the incubating cultures for 2 hr, resulting in a final concentration of 0.1 mM. Bacteria were collected by centrifugation at 6000×g for 20 min. The pellets were resuspended in 10 ml of lysis buffer (50 mM Tris, pH 7.5, 1% Triton-X, 150 mM NaCl, 5 mM MgCl₂, 1 mM DTT, 10 μg/ml leueptin, 10 μg/ml aprotinin, and 1 mM PMSF), sonicated, and lysates were spun at 14,000 rpm for 30 min at 4° C. The clarified bacterial lysate was diluted 1:100 and used for in situ binding studies. Paraformaldehyde-fixed retinal cryostat sections were incubated with diluted lysate overnight at 4° C., washed three times in TBS, blocked in 5% BSA in TBS with 0.05% Tween 20 for 1 hr at room temperature, and incubated with an anti-GST antibody (Immunology Consultants Laboratory, Newberg, Oreg.) and with the TUJ1 antibody (Babco) overnight at 4° C. as described (Dubreuil et al., 2002). Sections were washed in TBS and incubated for 2 hr at room temperature with Alexa Fluor 488 and 594-conjugated secondary antibodies (1:500, Molecular Probes).

### Viral Construction

cDNA encoding a modified form of the ADP ribosyl transferase C3 was generated by PCR from the pET-3a-C3 plasmid, generously provided by Dr. S. Narumiya (Kyoto University, Kyoto, Japan) (Kumagai et al., 1993), using the following primers: forward, 5′-TATGGCTAGCTATGC ACATACTTTCACAGAATT-3′ (SEQ ID NO: 17); reverse, 5′-CTATTTAAATATCATTGCTGTAATCATAATTTGTC-3′ (SEQ ID NO: 18). The encoded form (Fournier et al., 2001) and the dipeptide Met-Ala is attached to Ser¹. The cDNA was inserted into the AAV-MCS2-IGFP plasmid, developed by the Harvard Gene Therapy Initiative (HGTI). In addition, we ligated in-frame sequence encoding the first 10 amino acids of GAP-43 to target the protein to the cell membrance (Zuber et al., 1989; Liu et al., 1994). Gene expression was drive by a cytomegalovirus promoter; constructs also expressed enhanced green fluorescent protein (GFP) from an internal ribosome entry site (IRES). Controls were transfected with viruses expressing GFP alone. Virus production was performed at the HGTI Core Facility.

### Viral Transfections

To transfect RGCs, female Sprague Dawley rats (160-180 gm) were anesthetized with ketamine-xylazine, and the back of the eye was exposed intraorbitally. After withdrawing 10 μl of fluid from the eye, approximately 10¹¹ AAV particles in 10 μl of PBS were injected into the vitreous body using a micropipette, with care taken to avoid injury to the lens. Injections were done 2 weeks before optic nerve surgery to obtain high levels of transgene expression during the course of regeneration (Cheng et al., 2002).

### Results

Transfection of RGCs with AAV Expressing C3 ADP-Ribosyltransferase

We injected mature rats intravitreally with AAV expressing either GFP alone (AAV-GFP) or clostridium botulinum C3 ADP-ribosyltransferase (and GFP after an IRES: AAV-C3-IGFP) to inactivate RhoA. By virtue of AAV2 being neuron specific, and by virtue of RGC somata and axons being superficial in the retina, this method results in the transfection of approximately 75% of RGCs but little transfection of other cell types (DiPolo et al., 1998; Martin et al., 2002; Fischer et al., 2004). RT-PCR demonstrated a strong C3 signal in retinas transfected with AAV-C3-IGFP but none in controls transfected with AAV-GFP (data not shown). The high efficiency and specificity of transfection was verified by double-labeling studies showing the GFP reporter to be expressed in the same cells that express the RGC-specific tubulin isoform βIII tubulin. Using RBD-GST for in situ “pull-down assays” to detect RhoA in the active (GTP-bound) state (Dubreuil et al., 2003), we observed considerable binding in normal RGCs but much less in RGCs transfected with AAV-C3-IGFP. Thus, AAV transfection leads to strong transgene expression in RGCs, and in the case of C3 expression, this inactivates RhoA.

RhoA Inactivation and Macrophage Activation have Synergistic Effects In Vivo

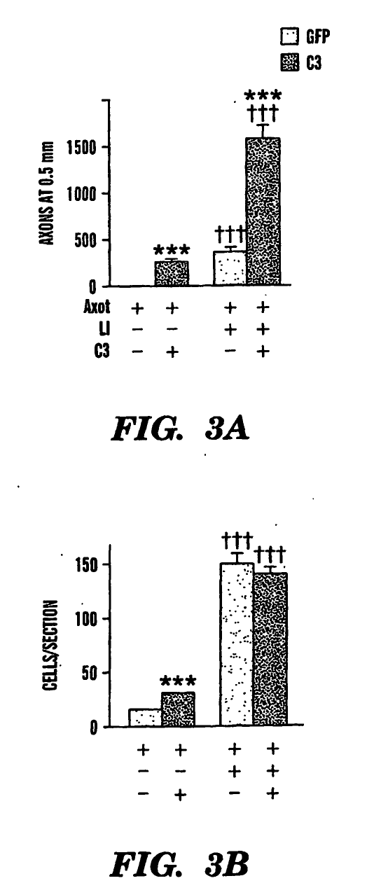

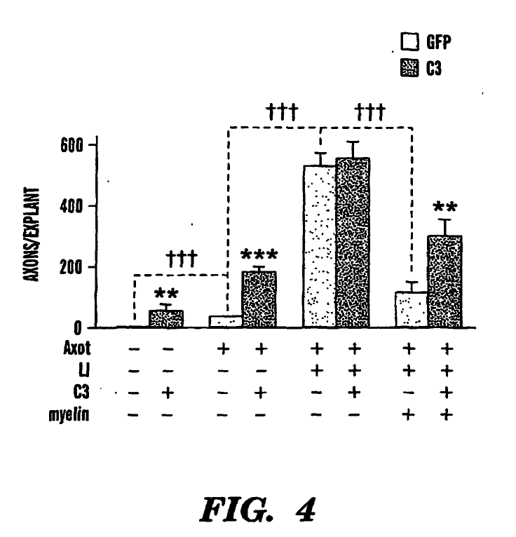

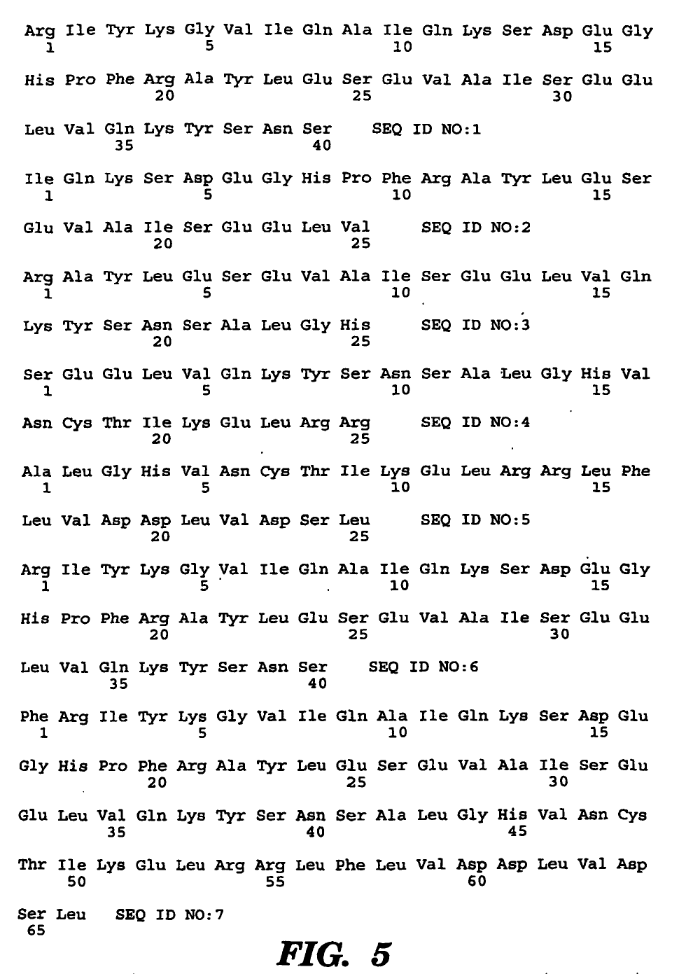

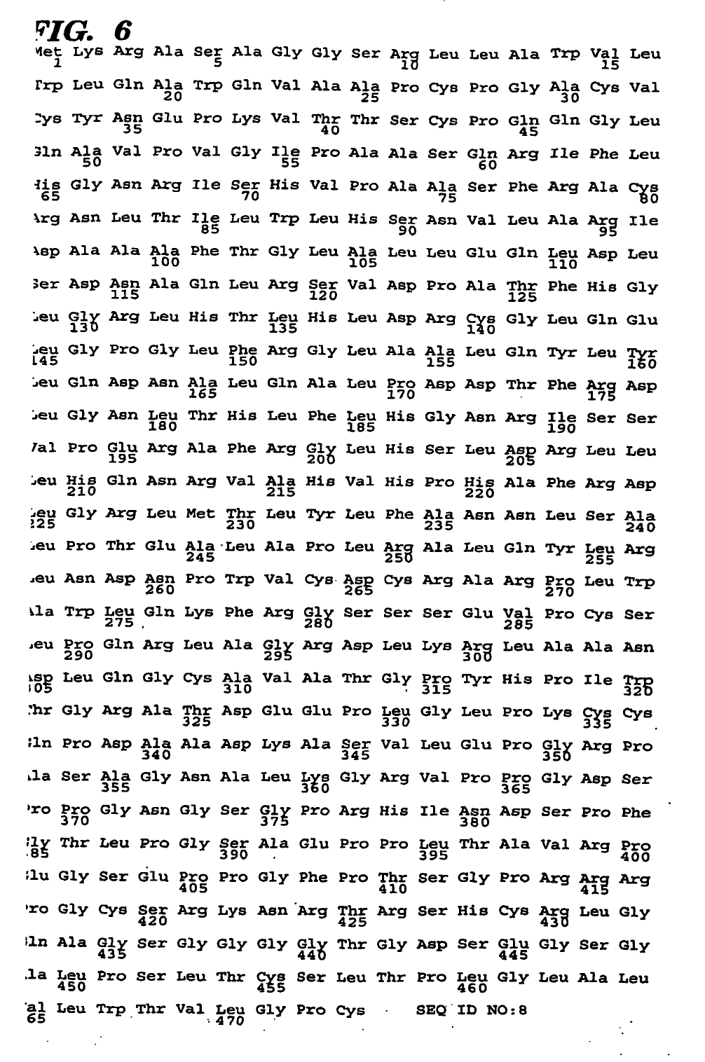

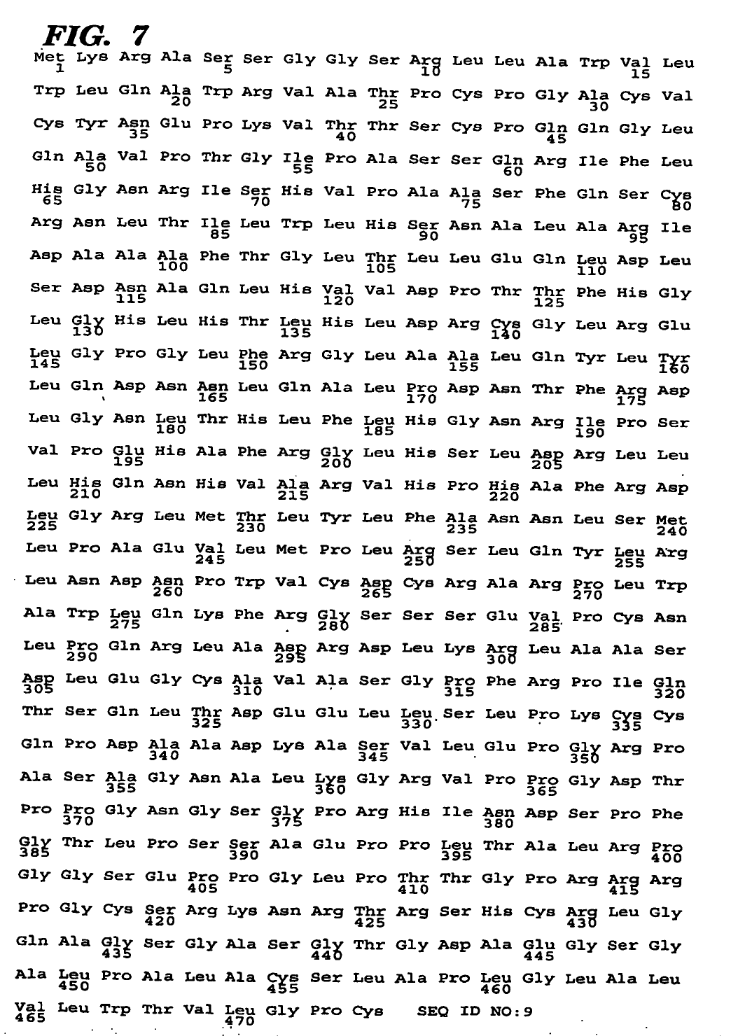

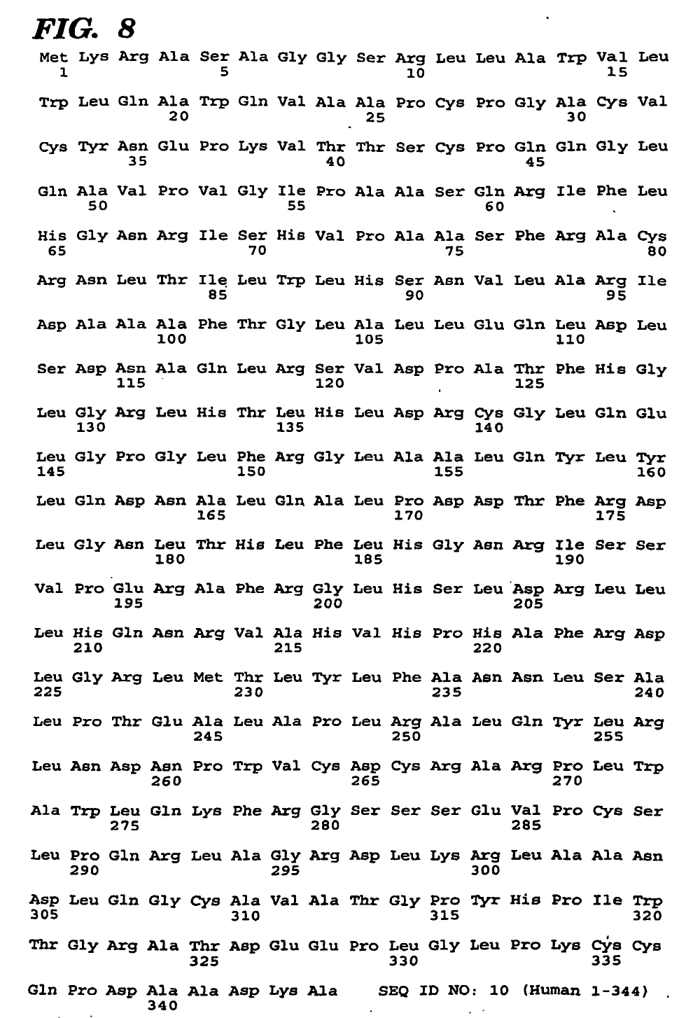

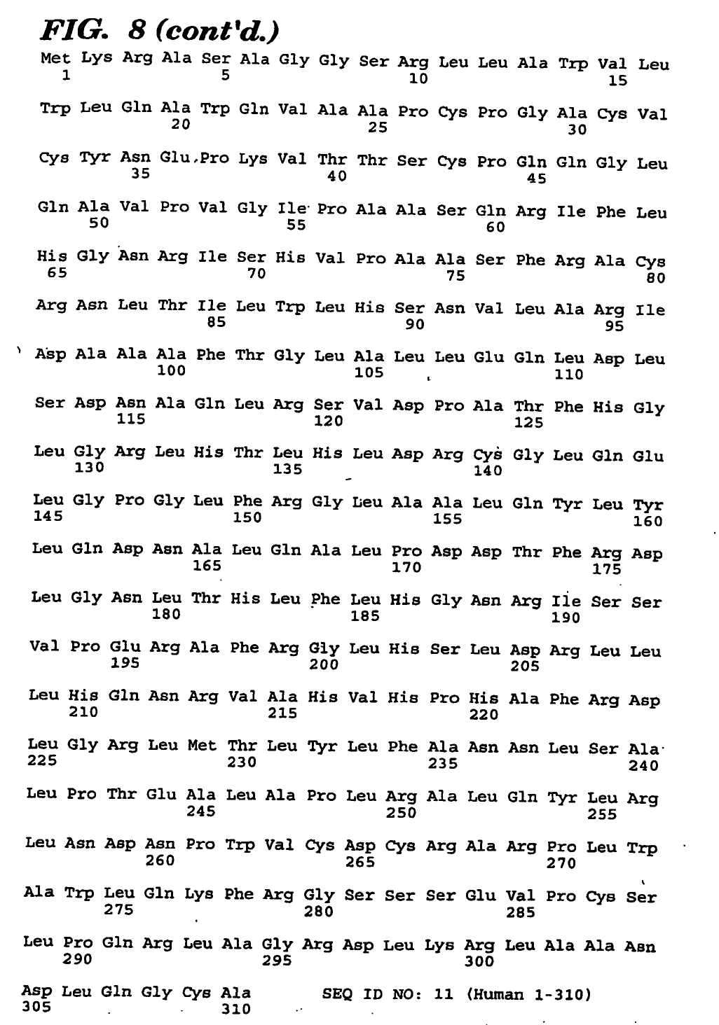

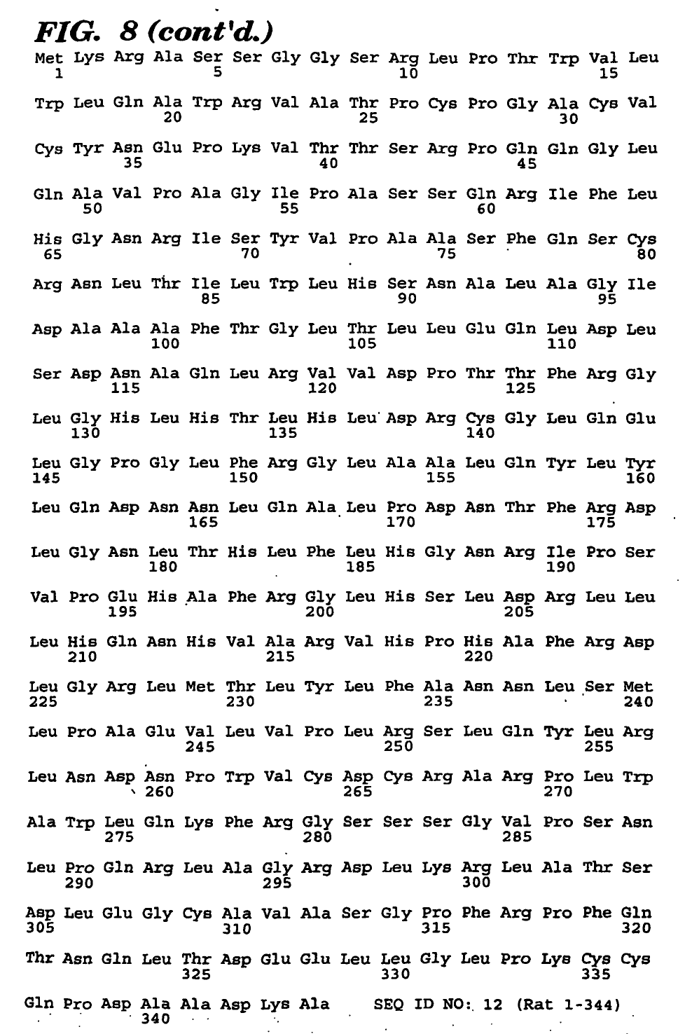

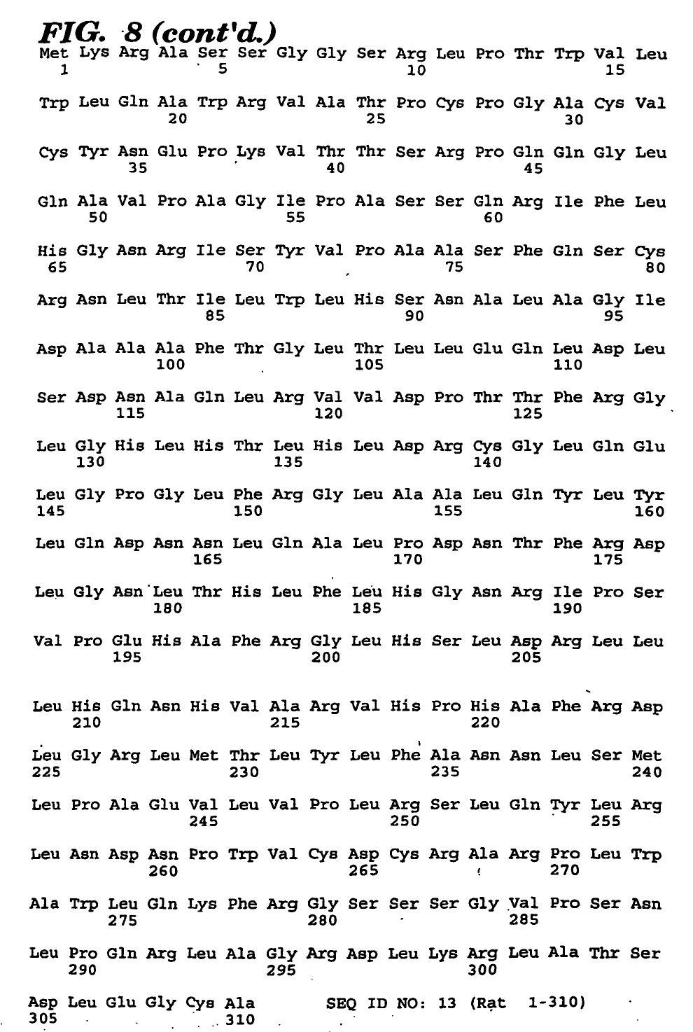

After allowing 2 weeks for transgenic C3 protein levels to become sufficiently high in RGCs, rats were re-anesthetized, and left optic nerve was crushed, and the lens was either injured or was left intact. Regeneration was evaluated 2 weeks later by GAP-43 immunostaining (Berry et al., 1996; Leon et al., 2000). As expected, AAV-GFP-transfected animals subjected to nerve crush alone showed no axons growing ≧500 μm beyond the lesion site 2 weeks after surgery (FIG. 8a), whereas similarly transfected animals with lens injury had, on average, approximately 400 axons extending ≧500 μm beyond the lesion site (FIG. 3a) (cf. Leon et al., 2000; Yin et al., 2003; Fischer et al., 2004). Even in the absence of lens injury, rats expressing C3 showed a modest number of axons passing through the lesion site; a higher percentage of these continued to extend ≧500 μm than was seen in GFP-expressing cases with lens injury, although the total number of axons reaching that criterion was lower (FIG. 3a). Combining C3 expression with lens injury resulted in unprecedented levels of axon regeneration. In every animal in this group, axon growth was so high as to obscure the discontinuity in GAP-43 immunostaining that is otherwise seen at the injury site. The number of axons extending ≧500 μm beyond the injury site was 4.5 times greater than after lens injury or C3 expression alone (FIG. 3a) (n=9; p<0.001) and higher than the effects of two added together. Thus, inactivation of RhoA and activation of the growth state of RGCs have synergistic effects in vivo.

### C3 Expression Enhances RGC Survival

RhoA inactivation by C3 has been reported to protect neurons and other cells from apoptotic cell death (Dubreuil et al., 2003). To investigate whether C3 affects RGC survival in vivo, we counted the number of TUJ1-positive cells from four to six cross sections through each retina (near the level of the optic nerve head) 2 weeks after nerve crush and lens injury. C3 expression increased RGC survival after nerve crush approximately twofold relative to controls expressing GFP alone but did not enhance the strong neuroprotective effects of lens injury any further (FIG. 3b).

### The Effects of C3 Expression on Growth State and Substrate

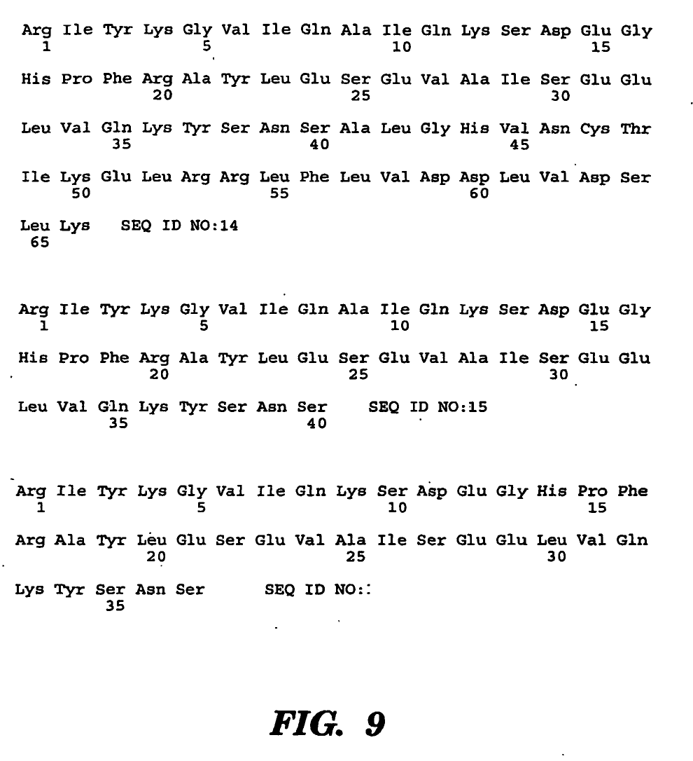

To investigate the effects of C3 expression in more detail, we examined the growth of retinal explants expressing C3 or GFP in culture. On a permissive laminin-poly-L-lysine substrate, control RGCs transfected with GFP showed almost no outgrowth, and C3 expression increased growth only slightly (FIG. 9) (p<0.001). Subjecting GFP-transfected RGCs to axotomy alone 4 d before-hand caused a moderate increase in regeneration compared with control RGCs (FIG. 9c,i) (p <0.001) (compare FIG. 1), and C3 transfection increased growth 4.6-fold when RGCs were in this state (p<0.001) (FIG. 9). Axotomy combined with lens injury increased growth 14-fold relative to RGCs subjected to axotomy alone, and this growth was not enhanced further C3 transfection (FIG. 9). Thus, when extrinsic inhibitors are absent, RhoA inactivation has only a small effect when the growth program of RGCs is not activated, a strong effect when the growth program is weakly activated by axotomy alone, but no additional effect when the growth program of RGCs is strongly activated.

When plated on a substrate containing myelin proteins, RGCs subjected to axotomy and lens injury showed far less growth than on poly-L-lysine-laminin (FIG. 9) (p<0.001) (cf. Fischer et al., 2004). Under these conditions, C3 expression increased the number of axons regenerating ≧50 μm 2.6-fold (FIG. 9) (p<0.02) and increased the number of axons growing ≧0.5 mm 3.8-fold (p=0.001; data not shown). Thus, when RGCs are in an active growth state, RhoA inactivation (by C3 expression) helps overcome the inhibitory effects of myelin.

### Discussion

RGCs in an active growth state can regenerate injured axons for considerable distances through the optic nerve, but their growth is still limited by inhibitory signals associated with myelin and the glial scar. Inactivating RhoA greatly potentiated the amount of growth that occurred when the growth state of neurons was activated. These findings support that clinically successful regeneration requires a multi-pronged approach.

The references cited herein are incorporated by reference in their entirety.

### REFERENCES

1. Aguayo A J, Rasminsky M, Bray G M, Carbonetto S, McKerracher L, Villegas-Perez M P, Vidal-Sanz M, Carter D A (1991) Degenerative and regenerative responses of injured neurons in the central nervous system of adult mammals. Philos Trans R Soc Lond B Biol Sci 331:337-343.
2. Bahr M, Vanselow J, Thanos S (1988) In vitro regeneration of adult rat ganglion cell axons from retinal explants. Exp Brain Res 73:393-401.
3. Bartlett J S, Samulski R J, McCown T J (1998) Selective and rapid uptake adeno-associated virus type 2 in brain. Hum Gene Ther 9:1181-1186.
4. Benowitz L I, Apostolides P J, Perrone-Bizzozero N, Finklestein S P, Zwiers H (1988) Anatomical distribution of the growth-associated protein GAP-43/B-50 in the adult rat brain. J Neurosci 8:339-352.
5. Berkelaar M, Clarke D B, Wang Y C, Bray G M, Aguayo A J (1994) Axotomy results in delayed death and apoptosis of retinal ganglion cells in adult rats. J Neurosci 14:4368-4374.
   6. Berry M, Carlile J, Hunter A (1996) Peripheral nerve explants grafted into the vitreous body of the eye promote the regeneration of retinal ganglion cell axons severed in the optic nerve. J. Neurocytol 25:147-170.
7. Bregman B S, Kunkel-Bagden E, Schnell L, Dai H N, Gao D, Schwab M E (1995) Recovery from spinal cord injury mediated by antibodies to neurite growth inhibitors. Nature 378:498-501.
8. Chen M S, Huber A B, van de Haar M E, Frank M, Schnell L, Spillmann A A, Christ F, Schwab M E (2000) Nogo-A is a myelin-associated neurite outgrowth inhibitor and an antigen for monoclonal antibody IN-1. Nature 403:434-439.
9. Cheng H, Cao Y, Olson L (1996) Spinal cord repair in adult paraplegic rats; partial restoration of hind limb function. Science 273:510-513.
10. Cheng L, Sapieha P, Kittlerova P, Hauswirth W W, Di Polo A (2002) TrkB gene transfer protects retinal ganglion cells from axotomy-induced death in vivo. J. Neurosci 22:3977-3986.
11. Cui Q, Yip H K, Zhao R C, So K F, Harvey A R (2003) Intraocular elevation of cyclic AMP potentiates ciliary neurotrophic factor-induced regeneration of adult rat retinal ganglion cell axons. Mol Cell Neurosci 22:49-61.
12. Di Polo A. et al., (1998) Prolonged delivery of brain-derived neurotrophic factor by adenovirus-infected Muller cells temporarily rescues injured retinal ganglion cells. Proc. Natl. Acad. Sci. USA 95:3978-3983.
13. Domeniconi M, Cao Z, Spencer T, Sivasankaran R, Wang K, Nikulina E, Kimura N, Cai H, Deng K, Gao Y, He Z, Filbin M (2002) Myelin-associated glycoprotein interacts with the nogo66 receptor to inhibit neurite outgrowth. Neuron 35:283.
14. Dubreuil et al. (2003) Rho activation patterns after spinal cord injury and the role of activated Rho in apoptosis in the central nervous system. J. Cell Biol. 162:233-243.
15. Fischer D, Pavlidis M, Thanos S (2000) Cataractogenic lens injury prevents traumatic ganglion cell death and promotes axonal regeneration both in vivo and in culture. Invest Opthalmol Vis Sci 41:3943-3954.
16. Fischer D, Heiduschka P, Thanos S (2001) Lens-injury-stimulated axonal regeneration throughout the optic pathway of adult rats. Exp Neurol 172:257-272.
17. Fischer D. et al., (2004) Counteracting the NOGO receptor enhances optic nerve regeneration if retinal ganglion cells are in an active growth state. J. Neurosci 24:1646-1651.
18. Fischer D. et al., (2004) Switching mature retinal ganglion cells to a robust growth state in vivo: gene expression and synergy with RhoA inactivation.
19. Fournier A E, GrandPre T, Strittmatter S M (2001) Identification of a receptor mediating Nogo-66 inhibition of axonal regeneration. Nature 409:341-346.
20. GrandPre T, Li S, Strittmatter S M (2002) Nogo-66 receptor antagonist peptide promotes axonal regeneration. Nature 417:547-551.
21. GrandPre T, Nakamura F, Vartanian T, Strittmatter S M (2000) Identification of the Nogo inhibitor of axon regeneration as a Reticulon protein. Nature 403:439-444.
22. Guest J D, Hesse D, Schnell L, Schwab M E, Bunge M B, Bunge R P (1997) Influence of IN-1 antibody and acidic FGF-fibrin glue on the response of injured corticospinal tract axons to human Schwann cell grafts. J Neurosci Res 50:888-905.
23. Kim J E, Liu B P, Yang X, Strittmatter S M (2003a) Recovery from spinal cord injury in mice lacking the Nogo-66 receptor. Program No 415.11, Abstract Viewer and Itinerary Planner. Washington, D.C.: Society Neuroscience, 2003 CD-ROM.
24. Kim J E, Li S, GrandPre T, Qiu D, Strittmatter S M (2003b) Axon regeneration in young adult mice lacking nogo-a/b. Neuron 38:187-199.
25. Kumagi N et al. (1993) ADP-ribosylation of rho p21 inhibits lysophosphatidic acid-induced protein tyrosine phosphorylation and phosphatidylinositol 3-kinase activation in cultured Swiss 3T3 cells. J. Biol. Chem. 268:24535-24538.
26. Lehmann M, Fournier A, Selles-Navarro I, Dergham P, Sebok A, Leclerc N, Tigyi G, McKerracher L (1999) Inactivation of Rho signaling pathway promotes CNS axon regeneration. J. Neurosci. 19:7537-7547.
27. Leon S, Yin, Y, Nguyen J, Irwing N, Benowitz K I (2000) Lens injury stimulates axon regeneration in the mature rat optic nerve. J Neurosci 20:4615-4626.
28. Li Y, Irwin N, Yin Y, Lanser M, Benowitz L I (2000) Axon regeneration in goldfish and rat retinal ganglion cells: differential responsiveness to carbohydrates and cAMP. J Neurosci 23:7830-7838.
29. Liu B P, Fournier A, GrandPre T, Strittmatter S M (2002) Myelin-Associated Glycoprotein as a Functional Ligand for the Nogo-66 Receptor. Science 27:27.
30. Liu Y. et al., (1994) Intracellular sorting of neuromodulin (GAP-43) mutants modified in the membrane targeting domain. J. Neurosci. 14:5807-5817.
31. Martin K R, Klein R L, Quigley H A (2002) Gene delivery to the eye using adeno-associated viral vectors. Methods 28:267-275.
32. McKeon R J, Hoke A, Silver J (1995) Injury-induced proteoglycans inhibit the potential for laminin-mediated axon growth on astrocytic scars. Exp Neurol 136:32-43.
33. McKerracher L, David S, Jackson D L, Kottis V, Dunn R J Braun P E (1994) Identification of myelin-associated glycoprotein as a major myelin-derived inhibitor of neurite growth. Neuron 13:805-811.
34. Moon, L D, Asher R A, Rhodes K E, Fawcett J W (2001) Regeneration of CNS axons back to their target following treatment of adult rat brain with chondroitinase ABC. Nat Neurosci 4:465-466.
35. Mukhopadhyay G, Doherty P, Walsh F S, Crocker P R, Filbin M T (1994) A novel role for myelin-associated glycoprotein as an inhibitor of axonal regeneration. Neuron 13:757-767.
36. Niederost B, Oertle T, Fritsche J, McKinney R A, Bandtlow C E (2002) Nogo-A and myelin-associated glycoprotein mediate neurite growth inhibition by antagonistic regulation of RhoA and RacI. J Neurosci 22:10368-10376.
37. Oertle T, van de Haar M E, Bandtlow C E, Robeva A, Burfeind P, Buss A, Huber A B, Simonen M, Schnell L, Brosamle C, Kaupmann K, Vallon R, Schwab M E (2003) Nogo-A inhibits neurite outgrowth and cell spreading with three discrete regions. J Neurosci 23:5393-5406.
38. Oster S F, Bodeker M O, He F, Sretavan D W (2003) Invariant Sema5A inhibition serves an ensheathing function during optic nerve development. Development 130:775-784.
39. Ramon y Cajal S (1991) Degeneration and Regeneration of the Nervous System. New York: Oxford University Press.
40. Schaden H. Stuermer C A, Bahr M (1994) GAP-43 immunoreactivity and axon regeneration in retinal ganglion cells of the rat. J Neurobiol 25:1570-1578.
41. Schnell L, Schneider R, Kolbeck R, Barde Y A, Schwab M E (1994) Neurotrophin-3 enhances sprouting of corticospinal tract during development and after adult spinal cord lesion. Nature 367:170-173.
42. Sicotte M, Tsatas O, Jeong S Y, Cai C Q, He Z, David S (2003) Immunization with myelin or recombinant Nogo-66/MAG in alum promotes axon regeneration and sprouting after corticospinal tract lesions in the spinal cord. Mol Cell Neurosci 23:251-263.
43. Simonen M, Pedersen V, Weinmann O, Schnell L, Buss A, Ledermann B, Christ F, Sansig G, van der Putten H, Schwab M E (2003) Systemic deletion of the myelin-associated outgrowth inhibitor nogo-a improves regenerative and plastic responses after spinal cord injury. Neuron 38:201-211.
44. Spillmann A A, Bandtlow C E, Lottspeich F, Keller F, Schwab M E (1998) Identification and characterization of a bovine neurite growth inhibitor (bNI-220). J Biol Chem 273:19283-19293.
45. Steward O, Zheng B, Tessier-Lavigne M (2003) False resurrections: distinguishing regenerated from spared axons in the injured central nervous system. J Comp Neurol 459:1-8.
46. Wang K C, Kim J A, Sivasankaran R, Segal R, He Z (2002a) p75 interacts with the Nogo receptor as a co-receptor for Nogo, MAG and OMgp. Nature 420:74-78.
47. Wang K C, Koprivica V, Kim J A, Sivasankaran R, Guo Y, Neve R L, He Z (2002b) Oligodendrocyte-myelin glycoprotein is a Nogo receptor ligand that inhibits neurite outgrowth. Nature 417:941-944.
48. Woolf C J (2003); No Nogo: now where to go? Neuron 38:153-156.
49. Yin Y, Cui Q, Li Y, Irwin N, Fischer D, Harvey A R, Benowitz L I (2003) Macrophage-derived factors stimulate optic nerve regeneration. J Neurosci 23:2284-2293.
50. Zheng B, Ho C, Li S, Keirstead H, Steward O, Tessier-Lavigne M (2003) Lack of enhanced spinal regeneration in nogo-deficient mice. Neuron 38:213-224.
51. Zuber et al. (1989) A membrane targeting signal in the amino terminus of the neuronal protein GAP-42. Nature 341: 345-348.

1. A Method for stimulating the axonal growth of central nervous system (CNS) neurons said method comprising the steps of:
a. contacting CNS neurons with an effective amount of an NgR antagonist; and
b. contacting CNS neurons with an effective amount of an agent that activates the growth pathway of CNS neurons.

2. The method of claim 1, wherein said CNS neurons are mammalian.

3. The method of claim 1, further comprising contacting CNS neurons with a cAMP modulator that increases the concentration of intracellular cAMP.

4. The method of claim 3, wherein the cAMP modulator is selected from the group consisting of: cAMP analogues, activators of G protein coupled receptors that activate cAMP, adenylate cyclase activators, calcium ionophores, and phosphodiesterase inhibitors.

5. The method of claim 1, wherein said agent that activates the growth pathway of CNS neurons is inosine.

6. The method of claim 1, wherein said agent that activates the growth pathway of CNS neurons is oncomodulin.

7. The method of claim 1, wherein said agent that activates the growth pathway of CNS neurons is a growth factor selected from the group consisting of TGF-β, NGF, BDNF, NT-3, CNTF, IL-6, and GDNF.

8. The method of claim 1, wherein said agent that activates the growth pathway of CNS neurons is a hexose.

9. The method of claim 8, wherein said hexose is selected from the group consisting D-mannose, gulose and glucose-6-phosphate.

10. The method of claim 1, wherein said NgR antagonist is an agent that binds to NgR thereby inhibiting signaling mediated by NgR.

11. The method of claim 1, wherein said NgR antagonist is an agent that inhibits the expression of NgR.

12. The method of claim 1, wherein said NgR antagonist is an agent that inhibits the activity of a downstream signaling molecule that is activated by NgR.

13. The method of claim 12, wherein the downstream signaling molecule is RhoA.

14. The method of claim 13, wherein said agent is clostridium botulinum C3 ADP-ribosyltransferase.

15. The method of claim 1, wherein said NgR antagonist is an antibody that binds NgR.

16. The method of claim 1, wherein said NgR antagonist is an antibody that binds a NgR ligand.

17. The method of claim 1, wherein said NgR antagonist is a peptide.

18. The method of claim 17, wherein said peptide comprises a peptide that binds to NgR selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

19. The method of claim 17, wherein said peptide comprises the amino acid residues of human NogoA set forth in SEQ ID NO: 14.

20. The method of claim 17, wherein said peptide comprises the amino acid residues of human NogoA forth in SEQ ID NO: 15.

21. The method of claim 17, wherein said peptide comprises the amino acid sequence of Nogo-66 set forth in SEQ ID NO: 16.

22. The method of claim 1, wherein said NgR antagonist is a soluble NgR protein.

23. The method of claim 22, wherein said soluble NgR protein comprises the amino acid sequence set forth in SEQ ID NO: 8 or in SEQ ID NO: 9.

24. The method of claim 22, wherein said soluble NgR protein is a soluble Nogo Receptor-1 polypeptide sequence selected from the group consisting of amino acid residues 26-344 of SEQ ID NO: 10; amino acid residues 26-310 of SEQ ID NO: 11; amino acid residues 26-344 of SEQ ID NO: 12; amino acid residues 27-344 of SEQ ID NO:12; and amino acid residues 27-310 of SEQ ID NO: 13.

25. The method of claim 1, wherein said NgR antagonist is a nucleic acid aptamer that binds to NgR.

26. The method of claim 1, wherein said NgR antagonist is a defective NgR encoded by a DNA.

27. The method of claim 26, wherein said defective NgR is a dominant-negative NgR.

28. The method of claim 1, wherein said NgR antagonist is clostridium botulinum C3 ADP-ribosyltransferase encoded by DNA.

29. The method of claim 26 or 28, wherein said DNA is contained in a viral vector whereby administration of said vector is a means for contacting CNS neurons with an effective amount of NgR antagonist.

30. The method of claim 36, wherein said viral vector is AAV.

31. A method for treating a neurological disorder in a patient comprising the steps of:
a. administering an effective amount of an NgR antagonist to said patient; and
b. administering an effective amount of an agent that activates the growth pathway of CNS neurons to said patient.

32. The method of claim 31, wherein said neurological disorder is selected from the group consisting of traumatic brain injury, stroke, cerebral aneurism, spinal cord injury, Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, opthalmoplegia, and retina or optic nerve damage.

33. The method of claim 31, wherein said agent that activates the growth pathway of CNS neurons is inosine.

34. The method of claim 31, wherein said agent that activates the growth pathway of CNS neurons is oncomodulin.

35. The method of claim 31, wherein said agent that activates the growth pathway of CNS neurons is a growth factor selected from the group consisting of TGF-β, NGF, BDNF, NT-3, CNTF, IL-6, and GDNF.

36. The method of claim 31, wherein said agent that activates the growth pathway of CNS neurons is a hexose.

37. The method of claim 36, wherein said hexose is selected from the group consisting D-mannose, gulose and glucose-6-phosphate.

38. The method of claim 31, wherein said NgR antagonist is an agent that binds to NgR thereby inhibiting signaling mediated by NgR.

39. The method of claim 31, wherein said NgR antagonist is an agent that inhibits the expression of NgR.

40. The method of claim 31, wherein said NgR antagonist is an agent that inhibits the activity of a downstream signaling molecule that is activated by NgR.

41. The method of claim 40, wherein the downstream signaling molecule is RhoA.

42. The method of claim 40, wherein said agent is clostridium botulinum C3 ADP-ribosyltransferase.

43. The method of claim 31, wherein said NgR antagonist is an antibody that binds NgR.

44. The method of claim 31, wherein said NgR antagonist is an antibody that binds a NgR ligand.

45. The method of claim 31, wherein said NgR antagonist is a peptide.

46. The method of claim 45, wherein said peptide comprises a peptide that binds to NgR selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

47. The method of claim 45, wherein said peptide comprises the amino acid residues of human NogoA set forth in SEQ ID NO: 14.

48. The method of claim 45, wherein said peptide comprises the amino acid residues of human NogoA forth in SEQ ID NO: 15.

49. The method of claim 45, wherein said peptide comprises the amino acid sequence of Nogo-66 set forth in SEQ ID NO: 16.

50. The method of claim 31, wherein said NgR antagonist is a soluble NgR protein.

51. The method of claim 50, wherein said soluble NgR protein comprises the amino acid sequence set forth in SEQ ID NO: 8 or in SEQ ID NO: 9.

52. The method of claim 50, wherein said soluble NgR protein is a soluble Nogo Receptor-1 polypeptide sequence selected from the group consisting of amino acid residues 26-344 of SEQ ID NO: 10; amino acid residues 26-310 of SEQ ID NO: 11; amino acid residues 26-344 of SEQ ID NO: 12; amino acid residues 27-344 of SEQ ID NO: 12; and amino acid residues 27-310 of SEQ ID NO: 13.

53. The method of claim 31, wherein said NgR antagonist is a nucleic acid aptamer that binds to NgR.

54. The method of claim 13, wherein said NgR antagonist is a defective NgR encoded by a DNA.

55. The method of claim 54, wherein said defective NgR is a dominant-negative NgR.

56. The method of claim 31, wherein said NgR antagonist is clostridium botulinum C3 ADP-ribosyltransferase encoded by a DNA.

57. The method of claim 54 or 56, wherein said DNA is contained in a viral vector whereby administration of said vector is a means for contacting CNS neurons with an effective amount of NgR antagonist.

58. The method of claim 57, wherein said viral vector is AAV.

59. A pharmaceutical composition comprising a NgR antagonist and an agent that activates the growth pathway of CNS neurons.

60. The pharmaceutical composition of claim 59, wherein the NgR antagonist is selected from the group consisting of an antibody, a peptide that binds to NgR, and a soluble NgR protein.

61. The pharmaceutical composition of claim 60, wherein the peptide is selected from the group consisting of the amino acid sequences set forth in SEQ ID NO: 1, SEQ ID NO: 2; SEQ ID NO: 3, SEQ ID NO: 4, SEQ ID NO: 5, SEQ ID NO: 6, SEQ ID NO: 7, SEQ ID NO: 14, SEQ ID NO: 15, and SEQ ID NO: 16.

62. The pharmaceutical composition of claim 60, wherein the soluble NgR protein is selected from the group consisting of the amino acid sequence set forth in SEQ ID NO: 8, the amino acid sequence set forth in SEQ ID NO: 9, amino acid residues 26-344 of SEQ ID NO: 10, amino acid residues 26-310 of SEQ ID NO: 11, amino acid residues 26-344 of SEQ ID NO: 12, amino acid residues 27-344 of SEQ ID NO: 12, and amino acid residues 27-310 of SEQ ID NO: 13.

63. The pharmaceutical composition of claim 59, wherein said composition is formulated for administration, the administration being selected from the group consisting of topical, pulmonary, internal topical, interdermal, parenteral, subcutaneous, intranasal, epidermal, ophthalmic, oral, intraventricular, and intrathecal.
